(19) United States
(12) Patent Application Publication
(10) Pub. No.: US 2003/0224940 A1
(43) Pub. Date: Dec. 4, 2003

(54) Alpha-sulfin-and alpha-sulfonamino acid amide derivatives

(76) Inventors: Martin Zeller, Baden (CH); Clemens Lamberth, Efringen-Kirchen (DE); Henry Szczepanski, Wallbach (CH); Alain De Mesmaeker, Kaenerkinden (CH)

Correspondence Address: SYNGENTA CROP PROTECTION , INC., PATENT AND TRADEMARK DEPARTMENT, 410 SWING ROAD, GREENSBORO, NC, 27409, US

(21) Appl. No.: 10/257,084

(22) PCT Filed: Apr. 11, 2001
(86) PCT No.: PCT/EP01/04206

(30) Foreign Application Priority Data
Apr. 13, 2000 (GB) 0009055.5

(51) Int. Cl. A01N 47/36; C07D 213/88; C07D 241/02

(52) U.S. Cl. 504/211; 544/59; 544/167; 544/406; 546/306; 564/39

(57) ABSTRACT

The invention relates to novel pesticidally active α-sulfin and α-sulfonamino acid amides of the general formula (I) including the optical isomers thereof and mixtures of such isomers, wherein n is a number zero or one; R₁, R₂, R₃, R₄, R₅, R₆ and R₇ have the meanings given in the specification, and R₈ is either hydrogen, (a), (b), (c), (d) or (e) wherein R₁₁, R₁₂, R₁₃, R₁₅ and R₁₇ are each independently hydrogen or C₁-C₄ alkyl, R₁₃ is C₄-C₁₂ alkyl, C₁-C₁₂ halogenalkyl; C₃-C₈ cycloalkyl; optionally substituted aryl or optionally substituted heteroaryl, R₁₆ is optionally substituted aryl or optionally substituted heteroaryl; and Z is oxygen, sulfur —CR₁₈R₁₉— or —NR_{2O}—, wherein R₁₈, R₁₉ and R₂₀ independently of each other are hydrogen or C₁-C₄ alkyl. The novel compounds possess plant-protecting properties and are suitable for protecting plants against infestation by phytopathogenic microorganisms.

The present invention relates to novel α-sulfin and α-sulfonamino acid amides of formula I below. It relates to the preparation of those substances and to agrochemical compositions comprising at least one of those compounds as active ingredient. The invention relates also to the preparation of the said compositions and to the use of the compounds or of the compositions in controlling or preventing the infestation of plants by phytopathogenic microorganisms, especially fungi.

The invention relates to α-sulfin- and α-sulfonamino acid amides of the general formula I

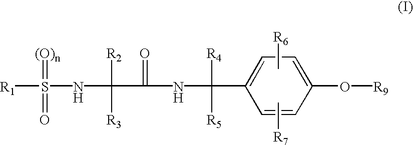

including the optical isomers thereof and mixtures of such isomers, wherein

n is a number zero or one;

R₁ is C₁-C₁₂alkyl, C₁-C₁₂alkyl substituted with C₁-C₄alkoxy, C₁-C₄alkylthio, C₁-C₄alkylsulfonyl, C₃-C₈cycloalkyl, cyano, C₁-C₆alkoxycarbonyl, C₃-C₆alkenyloxycarbonyl or C₃-C₆alkynyloxycarbonyl; C₃-C₈cycloalkyl; C₂-C₁₂alkenyl; C₂-C₁₂alkynyl; C₁-C₁₂halogenalkyl; or a group NR₉R₁₀ wherein R₉ and R₁₀ are each independenty of the other C₁-C₆alkyl, or together are tetra- or penta-methylene;

R₂ and R₃ are each independently hydrogen; C₁-C₈alkyl; C₁-C₈alkyl substituted with hydroxy, mercapto, C₁-C₄alkoxy or C₁-C₄alkylthio; C₃C₈alkenyl; C₃-C₈alkynyl; C₃-C₈cycloalkyl; C₃-C₈cycloalkyl-C₁-C₄alkyl; or the two groups R₂ and R₃ together with the carbon atom to which they are bonded form a three- to eight-membered hydrocarbon ring;

R₄ and R₅ are each independently hydrogen or C₁-C₄alkyl;

R₆ and R₇ are independently of each other hydrogen or an organic radical,

R₈ is either hydrogen

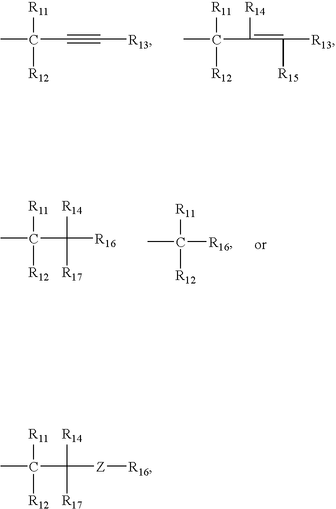

wherein

R₁₁, R₁₂, R₁₃, R₁₅ and R₁₇ are each independently hydrogen or C₁-C₄alkyl,

R₁₃ is C₄-C₁₂alkyl; C₁-C₁₂halogenalkyl; C₃-C₈cycloalkyl; optionally substituted aryl or optionally substituted heteroaryl,

R₁₆ is optionally substituted aryl or optionally substituted heteroaryl; and

Z is oxygen, sulfur —CR₁₈R₁₉— or —NR₂₀—, wherein R₁₈, R₁₉ and R₂₀ independently of each other are hydrogen or C₁-C₄alkyl.

In the above definition aryl includes aromatic hydrocarbon rings like phenyl, naphthyl, anthracenyl, phenanthrenyl, with phenyl being preferred.

Heteroaryl stands for aromatic ring systems comprising mono-, bi- or tricyclic systems wherein at least one oxygen, nitrogen or sulfur atom is present as a ring member. Examples are furyl, thienyl, pyrrolyl, imidazolyl, pyrazolyl, thiazolyl, isothiazolyl, oxazolyl, isoxazolyl, oxadiazolyl, thiadiazolyl, triazolyl, tetrazolyl, pyridyl, pyridazinyl, pyrimidinyl, pyrazinyl, triazinyl, tetrazinyl, indolyl, benzothiophenyl, benzofuranyl, benzimidazolyl, indazolyl, benzotriazolyl, benzothiazolyl, benzoxazolyl, quinolinyl, isoquinolinyl, phthalazinyl, quinoxalinyl, quinazolinyl, cinnolinyl and naphthyridinyl.

The above aryl and heteroaryl groups may carry one or more identical or different substituents. Normally not more than three substituents are present at the same time. Examples of substituents of aryl or heteroaryl groups are: alkyl, alkenyl, alkynyl, cycloalkyl, cycloalkyl-alkyl, phenyl and phenyl-alkyl, it being possible in turn for all of the preceding groups to carry one or more identical or different halogen atoms; alkoxy; alkenyloxy; alkynyloxy; alkoxyalkyl; halogenalkoxy, alkylthio; halogenalkylthio; alkyl sulfonyl; formyl; alkanoyl; hydroxy; halogen; cyano; nitro; amino; alkylamino: dialkyl amino; carboxy; alkoxycarbonyl; alkenyloxycarbonyl; alkynyloxycarbonyl.

In the above definitions “halogen” or the prefix “halo” includes fluorine, chlorine, bromine and iodine.

The alkyl, alkenyl and alkynyl radicals may be straight-chain or branched. This applies also to the alkyl, alkenyl or alkynyl parts of other alkyl-, alkenyl- or alkynyl-containing groups.

The organic radical in R₆ and R₇ indicates that practically every common substituent used in the art organic chemistry may be placed in the indicated position at the phenylene bridge member. Preferred are however the more frequently used radicals like C₁-C₈alkyl, C₂-C₈alkenyl, C₂-C₈alkynyl, C₃-C₈cycloalkyl, C₃-C₈cycloalkyl-C₁-C₄alkyl, C₁-C₈alkylthio, C₁-C₈alkylsulfonyl, C₁-C₈alkoxy, C₃-C₈alkenyloxy, C₃-C₈alkynyloxy, C₃-C₈cycloalkoxy, C₁-C₈alkoxy-C₁-C₄alkyl, C₁-C₈alkoxycarbonyl, C₃-C₈alkenyloxycarbonyl, C₃-C₈alkynyloxycarbonyl, C₁-C₈alkanoyl, C₁-C₈dialkylamino, C₁-C₈alkylamino wherein in each of the above radicals the alkyl, alkenyl, alkynyl or cycloalkyl groups may be partially or fully halogenated; carboxy, formyl, halogen, nitro, cyano, hydroxy or amino.

Depending upon the number of carbon atoms mentioned, alkyl on its own or as part of another substituent is to be understood as being, for example, methyl, ethyl, propyl, butyl, pentyl, hexyl, heptyl, octyl, nonyl, decyl, undecyl, dodecyl and the isomers thereof, for example isopropyl, isobutyl, tert-butyl or sec-butyl, isopentyl or tert-pentyl.

Cycloalkyl is, depending upon the number of carbon atoms mentioned, cyclopropyl, cyclobutyl, cyclopentyl, cyclohexyl, cycloheptyl or cyclooctyl.

Depending upon the number of carbon atoms mentioned, alkenyl as a group or as a structural element of other groups is to be understood as being, for example, ethenyl, allyl, 1-propenyl, buten-2-yl, buten-3-yl, penten-1-yl, penten-3-yl, hexen-1-yl, 4-methyl-3-pentenyl or 4-methyl-3-hexenyl.

Alkynyl as a group or as a structural element of other groups is, for example, ethynyl, propyn-1-yl, propyn-2-yl, butyn-1-yl, butyn-2-yl, 1-methyl-2-butynyl, hexyn-1-yl, 1-ethyl-2butynyl or octyn-1-yl.

A halogenalkyl group may contain one or more (identical or different) halogen atoms, and for example may stand for CHCl₂, CH₂F, CCl₃, CH₂Cl, CHF₂, CF₃, CH₂CH₂Br, C₂C₅, CH₂Br, CHClBr, CF₃CH₂, etc.

Where R₂ and R₃ together with the carbon atom to which they are attached form a hydrocarbon ring the ring corresponds to cyclopropane, cyclobutane, cyclopentane, cyclohexane, cycloheptane or cyclooctane

The presence of at least one asymmetric carbon atom and/or at least one asymmetric oxidized sulfur atom in the compounds of formula I means that the compounds may occur in optically isomeric forms. As a result of the presence of a possible aliphatic C═C double bond, geometric isomerism may also occur. Formula I is intended to include all those possible isomeric forms and mixtures thereof.

Preferred subgroups of compounds of formula I are those wherein

n is one; or

R₁ is C₁-C₁₂alkyl, C₁-C₁₂alkyl substituted with C₁-C₄alkoxy, C₁-C₄alkylthio, or C₁-C₄alkylsulfonyl; C₃-C₈cycloalkyl; C₂-C₁₂alkenyl; C₂-C₁₂alkynyl; C₁-C₁₂halogenalkyl; or a group NR₉R₁₀ wherein R₉ and R₁₀ are each independently of the other hydrogen or C₁-C₆alkyl, or together are tetra- or penta-methylene; or

R₁ is C₁-C₁₂alkyl, C₂-C₁₂alkenyl; C₁-C₁₂halogenalkyl; or a group NR₉R₁₀ wherein R₉ and R₁₀ are each independently of the other hydrogen or C₁-C₆alkyl; or

R₁ is C₁-C₄alkyl, C₂-C₄alkenyl; C₁-C₄halogenalkyl; or C₁-C₂dialkylamino; or

R₁ is C₁-C₄alkyl, vinyl; C₁-C₄halogenalkyl; or dimethylamino; or

R₂ is hydrogen and R₃ is C₁-C₈alkyl, C₁-C₈alkyl optionally substituted by hydroxy, C₁-C₄alkoxy, mercapto or C₁-C₄alkylthio; C₃-C₈alkenyl; C₃-C₈alkynyl; C₃-C₈cycloalkyl or C₃-C₈cycloalkyl-C₁-C₄alkyl; or

R₂ is hydrogen and R₃ is C₁-C₄alkyl; C₃-C₄alkenyl or cyclopropyl; or

R₂ is hydrogen and R₃ is C₃-C₄alkyl; allyl or cyclopropyl; or

R₂ is hydrogen and R₃ is isopropyl; or

R₄ is hydrogen, methyl or ethyl; or

R₄ is hydrogen or methyl; or

R₄ is hydrogen; or

R₅ is hydrogen or methyl; or

R₅ is hydrogen; or

R₆ is hydrogen, C₁-C₈alkyl, C₂-C₈alkenyl, C₂-C₈alkynyl, C₃-C₈cycloalkyl, C₃-C₈cycloalkyl-C₁-C₄alkyl, C₁-C₈alkylthio, C₁-C₈alkylsulfonyl, C₁-C₈alkoxy, C₃-C₈alkenyloxy, C₃-C₈alkynyloxy, C₃-C₈cycloalkoxy, C₁-C₈alkoxy-C₁-C₄alkyl, C₁-C₈alkoxycarbonyl, C₃-C₈alkenyloxycarbonyl, C₃-C₈alkynyloxycarbonyl, C₁-C₈alkanoyl, C₁-C₈dialkylamino, C₁-C₈alkylamino wherein in each of the above radicals the alkyl, alkenyl, alkynyl or cycloalkyl groups may be partially or fully halogenated; carboxy, formyl, halogen, nitro, cyano, hydroxy or amino; or

R₆ is hydrogen, C₁-C₈alkyl, C₁-C₈halogenalkyl, C₂-C₈alkenyl, C₂-C₈alkynyl, C₁-C₈alkylthio, C₁-C₈halogenalkylthio, C₁-C₈alkoxy, C₁-C₈halogenalkoxy, C₁-C₈alkoxy-C₁-C₄alkyl, C₁-C₈alkoxycarbonyl, C₁-C₈alkanoyl, formyl, halogen, nitro, cyano or hydroxy; or

R₆ is hydrogen, C₁-C₄alkyl, C₁-C₄halogenalkyl, C₁-C₄alkoxy, C₁-C₄alkoxycarbonyl, C₁-C₄alkanoyl, formyl, halogen, cyano or hydroxy; or

R₆ is hydrogen, C₁-C₄alkyl, C₁-C₄alkoxy, halogen or cyano; or

R₇ is hydrogen, C₁-C₈alkyl, C₂-C₈-alkenyl, C₂-C₈alkynyl, C₃-C₈cycloalkyl, C₃-C₈cycloalkyl-C₁-C₄alkyl, C₁-C₈alkylthio, C₁-C₈alkylsulfonyl, C₁-C₈alkoxy, C₃-C₈alkenyloxy, C₃-C₈alkynyloxy, C₃-C₈cycloalkoxy, C₁-C₈alkoxy-C₁-C₄alkyl, C₁-C₈alkoxycarbonyl, C₃-C₈alkenyloxycarbonyl, C₃-C₈alkynyloxycarbonyl, C₁-C₈alkanoyl, C₁-C₈dialkylamino, C₁-C₈alkylamino wherein in each of the above radicals the alkyl, alkenyl, alkynyl or cycloalkyl groups may be partially or fully halogenated; carboxy, formyl, halogen, nitro, cyano, hydroxy or amino; or

R₇ is hydrogen, C₁-C₄alkyl, C₁-C₄-halogenalkyl, C₁-C₄alkoxy, C₁-C₄alkoxycarbonyl, C₁-C₄alkanoyl, formyl, halogen, cyano or hydroxy; or

R₇ is hydrogen, C₁-C₄-alkyl, C₁-C₄alkoxy, halogen or cyano; or

R₇ is hydrogen; or

R₁₁, R₁₂, R₁₄, R₅ and R₁₇ are each independently hydrogen or methyl; or

R₁₁, R₁₂, R₁₄, R₁₅ and R₁₇ are each hydrogen; or

R₁₃ is C₄-C₁₂alkyl; C₁-C₁₂halogenalkyl; C₃-C₈cycloalkyl; optionally substituted aryl or optionally substituted heteroaryl consisting of one or two condensed five or six membered rings with 1 to 4 identical or different heteroatoms selected from oxygen, nitrogen or sulfur; or

R₁₃ is C₄-C₁₂alkyl; C₁-C₁₂halogenalkyl; C₃-C₈cycloalkyl; phenyl, naphthyl, furyl, thienyl, pyrrolyl, imidazolyl, pyrazolyl, thiazolyl, oxazolyl, isoxazolyl, thiazolyl, pyridyl, pyridazinyl, pyrimidinyl, pyrazinyl, triazinyl, indolyl, benzothiophenyl, benzofuranyl, benzimidazolyl, benzthiazolyl, benzoxazolyl or quinolyl with each of the aromatic ring being optionally substituted by 1 to 3 substituents selected from the group consisting of C ₁-C₈alkyl, C₂-C₈alkenyl, C₂-C₈alkynyl, C₃-C₈cycloalkyl, C₃-C₈cycloalkyl-C₁-C₄alkyl, phenyl, phenyl-C₁-C₄alkyl wherein the hydrogens of all these substituents may be optionally substituted by one or more identical or different halogen atoms; C ₁-C₈alkoxy, C₃-C₈alkenyloxy, C₃-C₈alkynyloxy, C₁-C₈alkoxy-C₁-C₄alkyl, C₁-C₈halogenalkoxy, C₁-C₈alkylthio, C₁-C₈halogenalkylthio, C₁-C₈alkylsulfonyl, formyl, C₂-C₈alkanoyl, hydroxy, halogen, cyano, nitro, amino, C₁-C₈alkylamino, C₁-C₈dialkylamino, carboxy, C₁-C₈alkoxycarbonyl, C₁C₈alkenyloxycarbonyl and C₁-C₈alkynyloxycarbonyl; or

R₁₃ is C₄-C₂alkyl; C₁-C₂halogenalkyl; C₃-C₈cycloalkyl; phenyl, naphthyl, thienyl, pyridyl, pyrimidinyl, triazinyl, or quinolyl with each of the aromatic ring being optionally substituted by 1 to 3 substituents selected from the group consisting of C ₁-C₈alkyl, C₂-C₈alkenyl, wherein the hydrogens of all these substituents may be optionally substituted by one or more identical or different halogen atoms; C₁-C₈alkoxy, C₁-C₈halogenalkoxy, C₁-C₈alkylthio, C₁-C₈halogenalkylthio, halogen, cyano, nitro and C₁-C₈alkoxycarbonyl; or

R₁₃ is C₄-C₈alkyl; C₁-C₆halogenalkyl; C₃-C₈cycloalkyl; phenyl, pyridyl, with each of the aromatic ring being optionally substituted by 1 to 3 substituents selected from the group consisting of C₁-C₈alkyl, C₁-C₈halogenalkyl, C₁-C₈alkoxy, C₁-C₈halogenalkoxy, C₁-C₈alkylthio, C₁-C₈halogenalkylthio, halogen, cyano, nitro and C₁-C₈alkoxycarbonyl; or

R₁₆ is optionally substituted aryl or optionally substituted heteroaryl consisting of one or two condensed five or six membered rings with 1 to 4 identical or different heteroatoms selected from oxygen, nitrogen or sulfur; or

R₁₆ is phenyl, naphthyl, furyl, thienyl, pyrrolyl, imidazolyl, pyrazolyl, thiazolyl, oxazolyl, isoxazolyl, thiazolyl, pyridyl, pyridazinyl, pyrimidinyl, pyrazinyl, triazinyl, indolyl, benzothiophenyl, benzofuranyl, benzimidazolyl, benzthiazolyl, benzoxazolyl or quinolyl with each of the aromatic ring being optionally substituted by 1 to 3 substituents selected from the group consisting of C₁-C₈alkyl, C₂-C₈alkenyl, C₂-C₈alkynyl, C₃-C₈cycloalkyl, C₃-C₈cycloalkyl-C₁-C₄-alkyl, phenyl, phenyl-C₁-C₄-alkyl wherein the hydrogens of all these substituents may be optionally substituted by one or more identical or different halogen atoms; C₁-C₈alkoxy, C₃-C₈alkenyloxy, C₃-Calkynyloxy, C₁-C₈-alkoxy-C₁-C₄alkyl, C₁-C₈halogenalkoxy, C₁-C₈alkylthio, C₁-C₈halogenalkylthio, C₁-C₈alkylsulfonyl, formyl, C₂-C₈alkanoyl, hydroxy, halogen, cyano, nitro, amino, C₁-C₈alkylamino, C₁-C₈dialkylamino, carboxy, C₁-C₈alkoxycarbonyl, C₁-C₈alkenyloxycarbonyl and C₁-C₈alkynyloxycarbonyl; or

R₁₆ is phenyl, naphthyl, thienyl, pyridyl, pyrimidinyl, triazinyl, or quinolyl with each of the aromatic ring being optionally substituted by 1 to 3 substituents selected from the group consisting of C₁-C₈alkyl, C₂-C₈alkenyl, wherein the hydrogens of all these substituents may be optionally substituted by one or more identical or different halogen atoms; C ₁-C₈-alkoxy, C₁-C₈halogenalkoxy, C₁-C₈alkylthio, C₁-C₈halogenalkylthio, halogen, cyano, nitro and C₁-C₈alkoxycarbonyl; or

R₁₆ is phenyl, pyridyl, with each of the aromatic ring being optionally substituted by 1 to 3 substituents selected from the group consisting of C₁-C₈alkyl, C₁-C₈halogenalkyl, C₁-C₈alkoxy, C₁-C₈halogenalkoxy, C₁-C₈alkylthio, C₁-C,halogenalkylthio, halogen, cyano, nitro and C₁-C₈alkoxycarbonyl; or

Z is oxygen, sulfur or —CH₂—; or

Z is oxygen.

Further preferred subgroups of the compounds of formula I are those wherein

1) R₁ is C₁-C₁₂alkyl, C₁-C₁₂alkyl substituted with C₁-C₄alkoxy, C₁-C₄alkylthio, or C₁-C₄alkylsulfonyl; C₃-C₈cycloalkyl, C₂-C₁₂alkenyl; C₂-C₁₂alkynyl; C₁-C₁₂halogenalkyl; or a group NR₉R₁₀ wherein R₉ and R₁₀ are each independently of the other hydrogen or C₁-C₆alkyl, or together are tetra- or penta-methylene;

R₂ is hydrogen and R₃ is C₁-C₈alkyl; C₁-C₈alkyl substituted with hydroxy, mercapto, C₁-C₄alkoxy or C₁-C₄alkylthio; C₃-C₈alkenyl; C₃-C₈alkynyl; C₃-C₈cycloalkyl; C₃-C₈cycloalkyl-C₁-C₄alkyl;

R₆ and R₇ are independently of each other hydrogen, C ₁-C₈alkyl, C₂-C₈alkenyl, C₂-C₈alkynyl, C₃-C₈cycloalkyl, C₃-C₈cycloalkyl-C₁-C₄alkyl, C₁-C₈alkylthio, C₁-C₈alkysulfonyl, C₁-C₈-alkoxy, C₃-C₈alkenyloxy, C₃-C₈alkynyloxy, C₃-C₈cycloalkoxy, C₁-C₈alkoxy-C₁-C₄alkyl, C₁-C₈alkoxycarbonyl, C₃-C₈alkenyloxycarbonyl, C₃-C₈alkynyloxycarbonyl, C₁-C₈alkanoyl, C₁-C₈dialkylamino, C₁-C₈alkylamino wherein in each of the above radicals the alkyl, alkenyl, alkynyl or cycloalkyl groups may be partially or fully halogenated; carboxy, formyl, halogen, nitro, cyano, hydroxy or amino;

R₁₃ is C₄-C₁₂alkyl; C₁-C₁₂halogenalkyl; C₃-C₈cycloalkyl; optionally substituted aryl or optionally substituted heteroaryl consisting of one or two condensed five or six membered rings with 1 to 4 identical or different heteroatoms selected from oxygen, nitrogen or sulfur; and

R₁₆ is optionally substituted aryl or optionally substituted heteroaryl consisting of one or two condensed five or six membered rings with 1 to 4 identical or different heteroatoms selected from oxygen, nitrogen or sulfur; or

2) n is one;

R₁ is C₁-C₁₂alkyl, C₂-C₁₂alkenyl; C₁-C₂halogenalkyl; or a group NR₉R₁₀ wherein R₉ and R₁₀ are each independently of the other hydrogen or C ₁-C₆alkyl;

R₂ is hydrogen and R₃ is C₁-C₄alkyl; C₃-C₄alkenyl or cyclopropyl;

R₄ is hydrogen, methyl or ethyl; or

R₅ is hydrogen or methyl;

R₆ is hydrogen, C₁-C₈alkyl, C₁-C₈halogenalkyl, C₂-C₈alkenyl, C₂-C₈alkynyl, C₁-C₈alkylthio, C₁-C₈halogenalkylthio, C₁-C₈alkoxy, C₁-C₈halogenalkoxy, C₁-C₈alkoxy-C₁-C₄alkyl, C₁-C₈alkoxycarbonyl, C₁-C₈alkanoyl, formyl, halogen, nitro, cyano or hydroxy;

R₇ is hydrogen, C₁-C₄alkyl, C₁-C₄halogenalkyl, C₁-C₄alkoxy, C₁-C₄alkoxycarbonyl, C₁-C₄alkanoyl, formyl, halogen, cyano or hydroxy; R₁₁, R₁₂, R₁₄, R₁₅ and R₁₇ are each independently hydrogen or methyl;

R₁₃ is C₄-C₁₂alkyl; C₁-C₁₂halogenalkyl; C₃-C₈cycloalkyl; phenyl, naphthyl, furyl, thienyl, pyrrolyl, imidazolyl, pyrazolyl, thiazolyl, oxazolyl, isoxazolyl, thiazolyl, pyridyl, pyridazinyl, pyrimidinyl, pyrazinyl, triazinyl, indolyl, benzothiophenyl, benzofuranyl, benzimidazolyl, benzthiazolyl, benzoxazolyl or quinolyl with each of the aromatic ring being optionally substituted by 1 to 3 substituents selected from the group consisting of C₁-C₈alkyl, C₂-C₈alkenyl, C₂-C₈alkynyl, C₃-C₆cycloalkyl, C₃-C₈cycloalkyl-C₁-C₄alkyl, phenyl, phenyl-C₁-C₄alkyl wherein the hydrogens of all these substituents may be optionally substituted by one or more identical or different halogen atoms; C₁-C₈alkoxy, C₃-C₈alkenyloxy, C₃-C₈alkynyloxy, C₁-C₈alkoxy-C₁-C₄alkyl, C₁-C₈halogenalkoxy, C₁-C₈alkylthio, C₁-C₈halogenalkylthio, C₁-C₈alkylsulfonyl, formyl, C₂-C₈alkanoyl, hydroxy, halogen, cyano, nitro, amino, C₁-C₈alkylamino, C₁-C₈dialkylamino, carboxy, C₁-C₈alkoxycarbonyl, C₁-C₈alkenyloxycarbonyl and C₁-C₈alkynyloxycarbonyl;

R₁₆ is phenyl, naphthyl, furyl, thienyl, pyrrolyl, imidazolyl, pyrazolyl, thiazolyl, oxazolyl, isoxazolyl, thiazolyl, pyridyl, pyridazinyl, pyrimidinyl, pyrazinyl, triazinyl, indolyl, benzothiophenyl, benzofuranyl, benzimidazolyl, benzthiazolyl, benzoxazolyl or quinolyl with each of the aromatic ring being optionally substituted by 1 to 3 substituents selected from the group consisting of C₁-C₈alkyl, C₂-C₈alkenyl, C₂-C₈alkynyl, C₃-C₈cycloalkyl, C₃-C₈cycloalkyl-C₁-C₄alkyl, phenyl, phenyl-C₁-C₄alkyl wherein the hydrogens of all these substituents may be optionally substituted by one or more identical or different halogen atoms; C₁-C₈alkoxy, C₃-C₈alkenyloxy, C₃-C₈alkynyloxy, C₁-C₈alkoxy-C₁-C₄alkyl, C₁-C₈halogenalkoxy, C₁-C₈-alkylthio, C₁-C₈halogenalkylthio, C₁-C₈alkylsulfonyl, formyl, C₂-C₈alkanoyl, hydroxy, halogen, cyano, nitro, amino, C₁-C₈alkylamino, C₁-C₈dialkylamino, carboxy, C₁-C₈alkoxycarbonyl, C₁-C₈alkenyloxycarbonyl and C₁-C₈alkynyloxycarbonyl; and

Z is oxygen, sulfur or —CH₂—; or

3) n is one;

R₁ is C₁-C₄alkyl, C₂-C₄alkenyl; C₁-C₄halogenalkyl; or C₁-C₂dialkylamino;

R₂ is hydrogen and R₃ is C₃-C₄alkyl; allyl or cyclopropyl;

R₄ is hydrogen or methyl;

R₅, R₁₁, R₁₂, R₁₄, R₁₅ and R₁₇ are each hydrogen;

R₆ is hydrogen, C₁-C₄alkyl, C₁-C₄halogenalkyl, C₁-C₄alkoxy, C₁-C₄alkoxycarbonyl, C₁-C₄alkanoyl, formyl, halogen, cyano or hydroxy;

R₇ is hydrogen, C₁-C₄alkyl, C₁-C₄alkoxy, halogen or cyano;

R₁₃ is C₄-C₁₂alkyl; C₁-C₁₂halogenalkyl; C₃-C₈cycloalkyl; phenyl, naphthyl, thienyl, pyridyl, pyrimidinyl, triazinyl, or quinolyl with each of the aromatic ring being optionally substituted by 1 to 3 substituents selected from the group consisting of C ₁-C₈alkyl, C₂-C₈alkenyl, wherein the hydrogens of all these substituents may be optionally substituted by one or more identical or different halogen atoms; C₁-C₈alkoxy, C₁-C₈halogenalkoxy, C₁-C₈alkylthio, C₁-C₈halogenalkylthio, halogen, cyano, nitro and C₁-C₈alkoxycarbonyl;

R₁₆ is phenyl, naphthyl, thienyl, pyridyl, pyrimidinyl, triazinyl, or quinolyl with each of the aromatic ring being optionally substituted by 1 to 3 substituents selected from the group consisting of C₁-C₈alkyl, C₂-C₈alkenyl, wherein the hydrogens of all these substituents may be optionally substituted by one or more identical or different halogen atoms; C₁-C₈alkoxy, C₁-C₈halogenalkoxy, C₁-C₈alkylthio, C₁-C₈halogenalkylthio, halogen, cyano, nitro and C₁-C₈alkoxycarbonyl; and

Z is oxygen, sulfur or —CH₂—; or

4) n is one;

R₁ is C₁-C₄alkyl, vinyl; C₁-C₄halogenalkyl; or dimethylamino;

R₂ is hydrogen and R₃ is isopropyl;

R₄, R₅, R₇, R₁₁, R₁₂, R₁₄, R₁₅ and R₁₇ are each hydrogen;

R₆ is hydrogen, Cl-C₄-alkyl, Cl-C₄alkoxy, halogen or cyano;

R₁₃ is C₄-C₈alkyl; C₁-C₆halogenalkyl; C₃-C₈cycloalkyl; phenyl, pyridyl, with each of the aromatic ring being optionally substituted by 1 to 3 substituents selected from the group consisting of C₁-C₈alkyl, C₁-C₈halogenalkyl, C₁-C₈alkoxy, C₁-C₈halogenalkoxy, C₁-C₈alkylthio, C₁-C₈halogenalkylthio, halogen, cyano, nitro and C₁-C₈alkoxycarbonyl;

R₁₆ is phenyl, pyridyl, with each of the aromatic ring being optionally substituted by 1 to 3 substituents selected from the group consisting of C ₁-C₈alkyl, C₁-C₈halogenalkyl, C₁-Calkoxy, C₁-C₈halogenalkoxy, C₁-C₈-alkylthio, C₁-C₈halogenalkylthio, halogen, cyano, nitro and C₁-C₈alkoxycarbonyl; and

Z is oxygen.

Preferred individual compounds are:

N-{4-[3-(4-chloro-phenyl)-prop-2-ynyloxy]-3-methoxy-benzyl}-2-ethanesulfonylamino-3-methyl-butyramide,

N-{4-[3-(4-fluoro-phenyl)-prop-2-ynyloxy]-3-methoxy-benzyl}-2-ethanesulfonylamino-3-methyl-butyramide,

N-{4-[3-(4-bromo-phenyl)-prop-2-ynyloxy]-3-methoxy-benzyl}-2-ethanesulfonylamino-3-methyl-butyramide,

N-{3-methoxy-4-[3-(p-tolyl)-prop-2-ynyloxy]-benzyl}-2-ethanesulfonylamino-3-methyl-butyramide,

N-[4-(3-cyclopropyl-prop-2-ynyloxy)-3-methoxy-benzyl]-2-ethanesulfonylamino-3-methyl-butyramide,

N-{4-[3-(4-chloro-phenyl)-prop-2-ynyloxy]-3-methoxy-benzyl}-2-methanesulfonylamino-3-methyl-butyramide,

N-{4-[3-(4-fluoro-phenyl)-prop-2-ynyloxy]-3-methoxy-benzyl}-2-methanesulfonylamino-3-methyl-butyramide,

N-{4-[3-(4-bromo-phenyl)-prop-2-ynyloxy]-3-methoxy-benzyl}-2-methanesulfonylamino-3-methyl-butyramide,

N-{3-methoxy-4-[3-(p-tolyl)-prop-2-ynyloxy]-benzyl}-2-methanesulfonylamino-3-methyl-butyramide, and

N-[4-(3-cyclopropyl-prop-2-ynyloxy)-3-methoxy-benzyl]-2-methanesulfonylamino-3-methyl-butyramide.

Certain α-sulfin- and α-sulfonamino acid derivatives having a different kind of structure have already been proposed for controlling plant-destructive fungi (for example in WO 95/030651, WO 97/14677, WO 98/38160 and WO 98/38161). The action of those preparations is not, however, satisfactory in all aspects of agricultural needs. Surprisingly, with the compound structure of formula I, new kinds of micro bicides having a high level of activity have been found.

The α-sulfin- and α-sulfonamino acid amides of formula I may be obtained according to one of the following processes:

a)

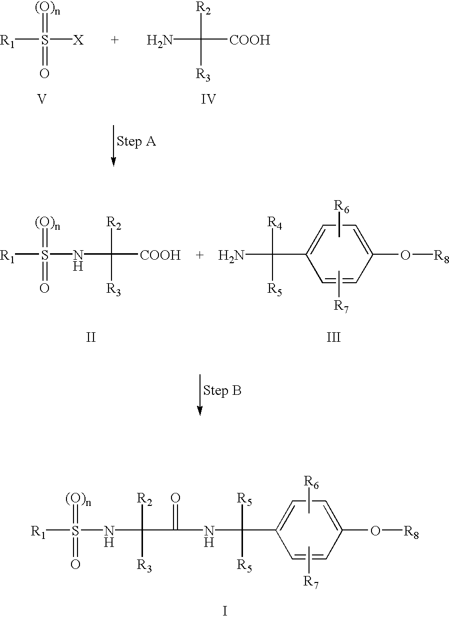

An amino acid of formula II or a carboxy-activated derivative of an amino acid of formula II wherein R₁, n, R₂ and R₃ are as defined for formula I is reacted with an amine of formula III wherein R₄, R₅, R₆, R₇ and R₈ are as defined above optionally in the presence of a base and optionally in the presence of a diluting agent (step B).

Carboxy-activated derivatives of the amino acid of formula II encompasses all compounds having an activated carboxyl group like an acid halide, such as an acid chloride, like symmetrical or mixed anhydrides, such as mixed anhydrides with O-alkylcarbonates, like activated esters, such as p-nitrophenylesters or N-hydroxysuccinimidesters, as well as in situ produced activated forms of the amino acid of formula II by condensating agents, such as dicyclohexylcarbodiimide, carbonyldiimidazol, benzotriazol-1-yloxy-tris (dimethylamino)-phosphonium hexafluorophosphate, O-benzotriazol-1-yl N,N,N′,N′-bis(penta methylene) -uronium hexafluorophosphate, O-benzotriazol-1-yl N,N,N′,N′-bis(tetramethylene)uronium hexafluorophosphate, O-benzotriazol-1-yl N,N,N′,N′-tetramethyluronium hexafluorophosfate or benzotriazol-1-yloxy-tripyrrolidino phosphonium hexafluorophosphate. The mixed anhydrides of the amino acids of the formula II may be prepared by reaction of an amino acid of formula II with chloroformic acid esters like chloroformic acid alkylesters, such as ethyl chloroformate or isobutyl chloroformate, optionally in the presence of an organic or inorganic base like a tertiary amine, such as triethylamine, N,N-diisopropyl-ethylamine, pyridine, N-methyl-piperidine or N-methyl-morpholine.

The present reaction is preferably performed in an inert solvent like aromatic, non-aromatic or halogenated hydrocarbons, such as chlorohydrocarbons e.g. dichloromethane or toluene; ketones, e.g. acetone; esters, e.g. ethyl acetate; amides, e.g. N,N -dimethyl-formamide; nitrites e.g. acetonitrile; or ethers e.g. diethylether, tert-butyl-methylether, dioxane or tetrahydrofurane or water. It is also possible to use mixtures of these solvents. The reaction is preformed optionally in the presence of an organic or inorganic base like a tertiary amine, e.g. triethylamine, N,N-diisopropyl-ethylamine, pyridine, N-methyl-piperidine or N-methyl-morpholine, like a metal hydroxide or a metal carbonate, preferentially an alkali hydroxide or an alkali carbonate, such as lithium hydroxide, sodium hydroxide or potassium hydroxide at temperatures ranging from −80° C. to +150° C., preferentially at temperatures ranging from −40° C. to +40° C.

The compounds of formula II may be prepared by reaction of an amino acid of formula IV where R₂ and R₃ are as defined for formula I with a sulfonyl halide or a sulfinyl halide of formula V where R₁ and n have the same meanings as defined above and where X is halide, preferentially chlorine or bromine (step A).

The reaction may be performed in an inert solvent like aromatic, non-aromatic or halogenated hydrocarbons, such as chlorohydrocarbons, e.g. dichloromethane or toluene; ketones, e.g. acetone; esters, e.g. ethyl acetate; ethers, e.g. diethylether, tert-butyl-methylether, dioxane or tetrahydrofurane or water. It is also possible to use mixtures of these solvents. The reaction is performed optionally in the presence of an organic or inorganic base like a tertiary amine, such as triethylamine, N,N-diisopropyl-ethylamine, pyridine, N-methyl-piperidine or N-methyl-morpholine, like a metal hydroxide or a metal carbonate, preferentially an alkali hydroxide or an alkali carbonate, such as lithium hydroxide, sodium hydroxide or potassium hydroxide at temperatures ranging from −80° C. to +150° C., preferentially at temperatures ranging from −40° C. to +40° C.

b)

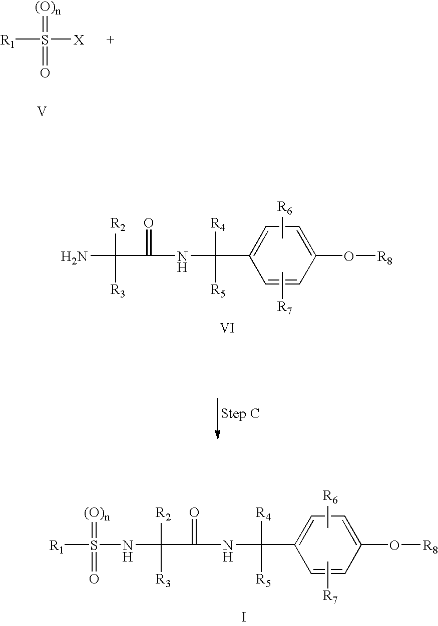

The compounds of formula I may also be prepared by reaction of an amino acid derivative of formula VI wherein R₂, R₃, R₄, R₅, R₆, R₇ and R₈ are as defined for formula I with a sulfonyl halide or a sulfinyl halide of formula V wherein R₁ and n are as defined for formula I and X is halide, preferentially chlorine or bromine (step C). The reaction is performed in the same manner as described for step A.

c)

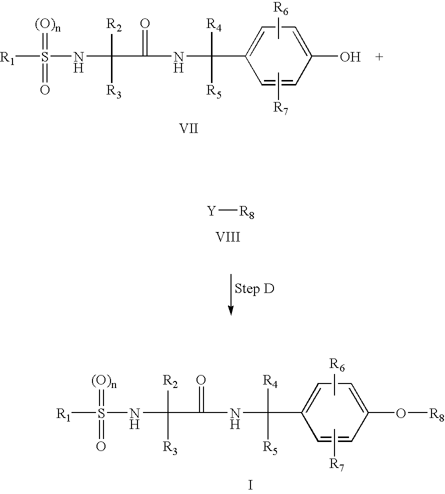

The compounds of formula I may also be prepared by reaction of a phenol of formula VII wherein R₁, n, R₂, R₃, R₄, R₅, R₆ and R₇ are as defined for formula I with a compound of formula VIII wherein R₈ is as defined for formula I and Y is a leaving group like a halide such as a chloride or bromide or a sulfonic ester such as a tosylate, mesylate or triflate (step D).

The reaction may be performed in an inert solvent like aromatic, non-aromatic or halogenated hydrocarbons, such as chlorohydrocarbons e.g. dichloromethane or toluene; ketones e.g. acetone or 2-butanone; esters, e.g. ethyl acetate; ethers, e.g. diethylether, tert-butyl-methylether, dioxane or tetrahydrofurane, amides, e.g. dimethylformamide, nitriles, e.g. acetonitrile, alcohols, e.g. methanol, ethanol, isopropanol, n-butanol or tert-butanol, sulfoxides e.g. dimethylsulfoxide or water. It is also possible to use mixtures of these solvents. The reaction is performed optionally in the presence of an organic or inorganic base like a tertiary amine, such as triethylamine, N,N-diisopropyl-ethylamine, pyridine, N-methyl-piperidine or N-methyl-morpholine, like a metal hydroxide, a metal carbonate or a metal alkoxide, preferentially an alkali hydroxide, an alkali carbonate or an alkali alkoxide, such as lithium hydroxide, sodium hydroxide, potassium hydroxide, sodium carbonate, potassium carbonate, sodium methoxide, potassium methoxide, sodium ethoxide, potassium ethoxide, sodium tert-butoxide or potassium tert-butoxide at temperatures ranging from −80° C. to +200° C., preferentially at temperatures ranging from 0° C. to +120° C.

d)

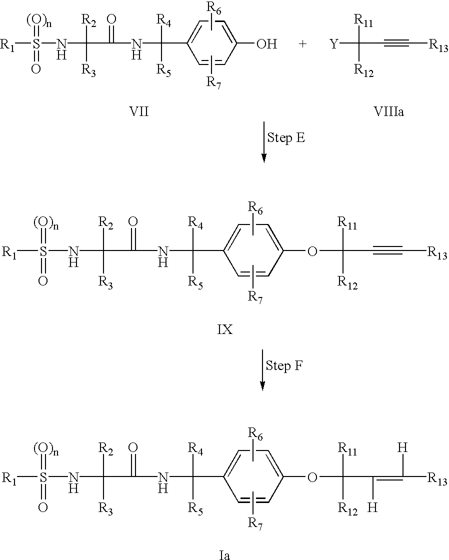

The compounds of formula Ia may also be prepared via formula IX wherein R₁, n, R₂, R₃, R₄, R₅, R₆, R₇, R₁₁, R₁₂ and R₁₃ are defined for formula I by reacting of a phenol of formula VII wherein R₁, n, R₂, R₃, R₄, R₅, R₆ and R₇ are as defined for formula I with compounds of formula VIIIa wherein R₁₁, R₁₂ and R₁₃ are as defined for formula I and Y is a leaving group like a halide such as a chloride or bromide or a sulfonic ester such as a tosylate, mesylate or triflate (step E).

The reaction is performed in the same manner as described for step D.

The compounds of formula Ia R₁₁, R₁₂ and R₁₃ is as defined for formula I may be prepared by reaction of compounds of formula IX with hydrogen.

The reaction is performed in a solvent like ethers, e.g. diethylether, dioxane or tetrahydrofuran, or like alcohols, e.g. methanol or ethanol, or water in the presense of transition metals or transition metal salts, e.g. nickel, cobalt, palladium, platinium or rhodium, optionally in the presense of bases, e.g. ammonia, or in the presense of salts, e.g. barium sulfate, at temperatures ranging from −20° C. to +160° C. and at pressures ranging from 1 to 200 bar.

aa) The intermediate amines of formula III may be obtained by one of the following processes:

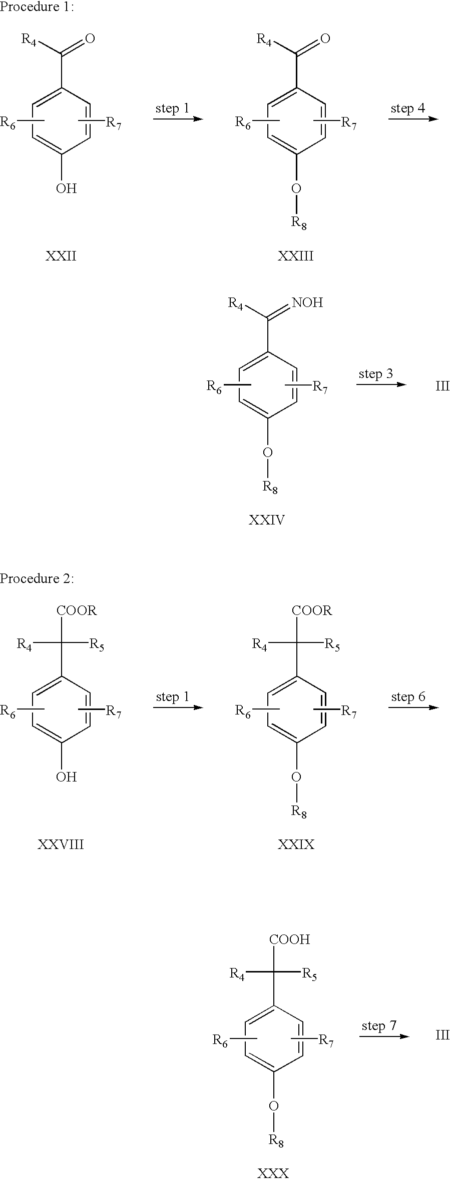

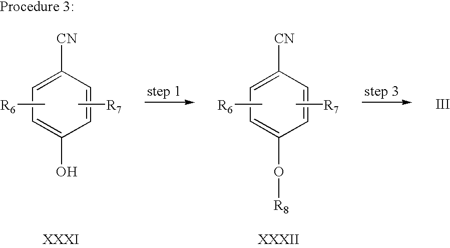

Step 1 is the alkylation of a phenol with a compound of formula VIII. The reaction is performed in the same manner as described for procedure c).

Step 3 is the reduction of an unsaturated nitrogen-compound. This reaction is performed in a solvent like an ether, e.g. diethylether, dioxane or tetrahydrofuran, or an alcohol, e.g. methanol, ethanol or isopropanol, with borohydride, with a boron-complex, e.g. the complex of borohydride with tetrahyrofuran, with an alkaliborohydride, with an alkalialuminiumhydride, e.g. lithiumaluminiumhydride, with aluminiumhydride, with an aluminiumalkoxyhydride or with hydrogen optionally in the presence of a transition metal, a transition metal salt or a transition metal complex, e.g. nickel, cobalt, palladium, platinium or rhodium at temperatures ranging from −50° C. to +200° C.

Step 4 is the reaction of an aldehyde or a ketone of formula with hydroxylamine or with a salt of hydroxylamine. This reaction is performed in a solvent like an alcohol, e.g. methanol, ethanol or isopropanol, like an ether, e.g. diethylether, dioxane or tetrahydrofuran, like an amide, e.g. dimethylformamide, or in water or in a mixture of these solvents optionally in the presence of an organic or inorganic base like a tertiary amine, e.g. triethylamine, like a heterocyclic compound containing nitrogen, e.g. pyridine, or like an alkalicarbonate, e.g. sodium carbonate or potassium carbonate, at temperatures ranging from −20° C. to +150° C.

Step 6 is the hydrolysis of an alkyl ester. This reaction is performed in a solvent like an alcohol, e.g. methanol, ethanol or isopropanol, like an ether, e.g. diethylether, dioxane or tetrahydrofuran, like a halogenated hydrocarbon, e.g. dichloromethane, or water or in a mixture of these solvents optionally in the presence of an alkali hydroxide, e.g. lithium hydroxide, sodium hydroxide or potassium hydroxide, or optionally in the presence of an acid, e.g. hydrogen chloride, sulfuric acid or trifluoroacetic acid at temperatures ranging from −20° C. to +160° C.

Step 7 is the reaction of a carboxylic acid or the activated form of this carboxylic acid with hydrogen azide or an azide-salt. An activated form of a carboxylic acid can be the acid halogenide, e.g. acid chloride, a symmetric or a mixed anhydride. Azide-salts can be alkali azides, e.g. sodium azide. The reaction is performed in a solvent like a hydrocarbon, e.g. toluene or xylene, like a halogenated hydrocarbon, e.g. chloroform, like an ether, e.g. dioxane, like a ketone, e.g. acetone or 2-butanone, like an alcohol, e.g. methanol, ethanol or tert-butanol, or water or in a mixture of these solvents optionally in the presence of an acid like an inorganic acid, e.g. sulfuric acid or hydrogen chloride at temperatures ranging from −40° C. to +200° C.

In a preferred form the compounds of formula XXVI are prepared starting from compounds of the formula XXV by applying step 5 and step 1 in the same pot.

bb) Amines of formula VI can be obtained by the following process:

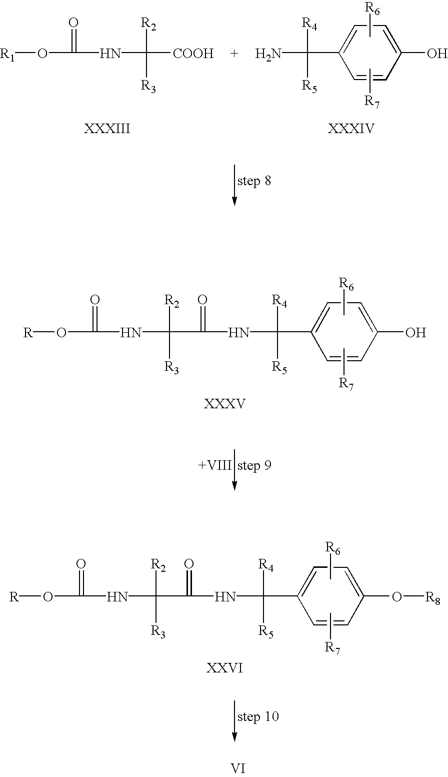

wherein R is lower alkyl or optionally substituted benzyl.

Step 8 is the amidation of an carbamate-protected amino acid of formula XXXIII with an amine of formula XXXIV. The reaction is performed in the same manner as described for step A.

Step 9 is the alkylation of a phenol of formula XXXV with an compound of formula VIII. The reaction is performed in the same manner as described for step D.

Step 10 is the hydrolysis of a carbamate of formula XXXVI. The reaction is performed in a solvent like hydrocarbons, e.g. toluene, like halogenated hydrocarbons, e.g. dichloromethane, like ketones, e.g. acetone, like esters, e.g. ethyl acetate, like ethers, e.g. dioxane or tetrahydrofuran, or like water or in mixtures of these solvents optionally in the presence of an organic acid like carboxylic acid, e.g. trifluoroacetic acid, or like a sulfonic acid, e.g. methanesulfonic acid or toluenesulfonic acid, or in the presence of an inorganic acid, e.g. hydrogen chloride or sulfuric acid, at temperatures ranging from −40° C. to +160° C.

The compounds of formula I are oils or solids at room temperature and are distinguished by valuable microbiocidal properties. They can be used in the agricultural sector or related fields preventively and curatively in the control of plant-destructive micro organisms. The compounds of formula I according to the invention are distinguished at low rates of concentration not only by outstanding microbicidal, especially fungicidal, activity but also by being especially well tolerated by plants.

Surprisingly, it has now been found that the compounds of formula I have for practical purposes a very advantageous biocidal spectrum in the control of phytopathogenic microorganisms, especially fungi. With the compounds of formula I it is possible to inhibit or destroy phytopathogenic microorganisms that occur on various crops of useful plants or on parts of such plants (fruit, blossom, leaves, stems, tubers, roots), while parts of the plants which grow later also remain protected, for example, against phyto pathogenic fungi.

The novel compounds of formula I prove to be effective against specific genera of the fungus class Fungi imperfecti (e.g. Cercospora), Basidio mycetes (e.g. Puccinia) and Ascomycetes (e.g. Erysiphe and Venturia) and especially against Oomycetes (e.g. Plasmopara, Perono spora, Pythium and Phytophthora). They therefore represent in plant protection a valuable addition to the compositions for controlling phyto pathogenic fungi. The compounds of formula I can also be used as dressings for protect ing seed (fruit, tubers, grains) and plant cuttings from fungal infections and against phyto pathogenic fungi that occur in the soil.

The invention relates also to compositions comprising compounds of formula I as active ingredient, especially plant-protecting compositions, and to the use thereof in the agricultural sector or related fields.

In addition, the present invention includes the preparation of those compositions, wherein the active ingredient is homogeneously mixed with one or more of the substances or groups of substances described herein. Also included is a method of treating plants which is distinguished by the application of the novel compounds of formula I or of the novel compositions.

Target crops to be protected within the scope of this invention comprise, for example, the following species of plants: cereals (wheat, barley, rye, oats, rice, maize, sorghum and related species); beet (sugar beet and fodder beet); pomes, stone fruit and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucurbitaceae (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamon, camphor) and plants such as tobacco, nuts, coffee, sugar cane, tea, pepper, vines, hops, bananas and natural rubber plants, and also ornamentals.

The compounds of formula I are normally used in the form of compositions and can be applied to the area or plant to be treated simultaneously or in succession with other active ingredients. Those other active ingredients may be fertilisers, micronutrient donors or other preparations that influence plant growth. It is also possible to use selective herbi cides or insecticides, fungicides, bactericides, nemati cides, molluscicides or mixtures of several of those preparations, if desired together with further carriers, surfactants or other application-promoting adjuvants customarily employed in formulation technology.

The compounds of formula I can be mixed with other fungicides, resulting in some cases in unexpected synergistic activities.

Mixing components which are particularly preferred are azoles such as azoles, such as azaconazole, bitertanol, bromuconazole, cyproconazole, difenoconazole, diniconazole, epoxiconazole, fenbuconazole, fluquinconazole, flusilazole, flutriafol, hexaconazole, imazalil, imibenconazole, ipconazole, metconazole, myclobutanil, pefurazoate, penconazole, pyrifenox, prochloraz, propiconazole, tebuconazole, tetraconazole, triadimefon, triadimenol, triflumizole, triticonazole; pyrimidinyl carbinols, such as ancymidol, fenarimol, nuarimol; 2-amino-pyrimidines, such as bupirimate, dimethirimol, ethirimol; morpholines, such as dodemorph, fenpropidine, fenpropimorph, spiroxamine, tridemorph; anilinopyrimidines, such as cyprodinil, mepanipyrim, pyrimethanil; pyrroles, such as fenpiclonil, fludioxonil; phenylamides, such as benalaxyl, furalaxyl, metalaxyl, R-metalaxyl, ofurace, oxadixyl; benzimidazoles, such as benomyl, carbendazim, debacarb, fuberidazole, thiabendazole; dicarboximides, such as chlozolinate, dichlozoline, iprodione, myclozoline, procymidone, vinclozolin; carboxamides, such as carboxin, fenturam, flutolanil, mepronil, oxycarboxin, thifluzamide; guanidines, such as guazatine, dodine, iminoctadine; strobilurines, such as azoxystrobin, kresoxim-methyl, metominostrobin, SSF-129, CGA 279202 (trifloxystrobin), picoxystrobin; dithiocarbamates, such as ferbam, mancozeb, maneb, metiram, propineb, thiram, zineb, ziram; N-halogenmethylthiophthalimides, such as captafol, captan, dichlofluanid, fluoromide, folpet, tolyfluanid; Cu compounds, such as Bordeaux mixture, copper hydroxide, copper oxychloride, copper sulfate, cuprous oxide, mancopper, oxine-copper; nitrophenol derivatives, such as dinocap, nitrothal-isopropyl; organo-P derivatives, such as edifenphos, iprobenphos, isoprothiolane, phosdiphen, pyrazophos, tolclofos-methyl; various, such as AC 382042, acibenzolar-S-methyl, anilazine, blasticidin-S, quinomethionat, chloroneb, chlorothalonil, cymoxanil, dichlone, diclomezine, dicloran, diethofencarb, dimethomorph, dithianon, etridiazole, famoxadone, fenamidone, fenhexamid, fentin, ferimzone, fluazinam, flusulfamide, fosetyl-aluminium, hymexazol, IKF-916, iprovalicarb, kasugamycin, methasulfocarb, MON65500, pencycuron, phthalide, polyoxins, probenazole, propamocarb, pyroquilon, quinoxyfen, quintozene, RH-7281, RPA 407213, BAS 50001F, sulfur, SYP-Z071, triazoxide, tricyclazole, triforine, validamycin.

Suitable carriers and surfactants may be solid or liquid and correspond to the substances ordinarily employed in formulation technology, such as e.g. natural or regenerated mineral substances, solvents, dispersants, wetting agents, tackifiers, thickeners, binders or fertilisers. Such carriers and additives are described, for example, in WO 95/30651.

A preferred method of applying a compound of formula I, or an agrochemical composition comprising at least one of those compounds, is application to the foliage (foliar application), the frequency and the rate of application depending upon the risk of infesta tion by the pathogen in question. The compounds of formula I may also be applied to seed grains (coating) either by impregnating the grains with a liquid formulation of the active ingredient or by coating them with a solid formulation.

The compounds of formula I are used in unmodified form or, preferably, together with the adjuvants conventionally employed in formulation technology, and are for that purpose advantageously formulated in known manner e.g. into emulsifiable concentrates, coatable pastes, directly sprayable or dilutable solutions, dilute emulsions, wettable powders, soluble powders, dusts, granules, and by encapsulation in e.g. polymer substances. As with the nature of the compositions, the methods of application, such as spraying, atomising, dusting, scattering, coating or pouring, are chosen in accordance with the intended objectives and the prevailing circumstances.

Advantageous rates of application are normally from 1 g to 2 kg of active ingredient (a.i.) per hectare (ha), preferably from 10 g to 1 kg a.i./ha, especially from 25 g to 750 g a.i./ha. When used as seed dressings, rates of from 0.001 g to 1.0 g of active ingredient per kg of seed are advantageously used.

The formulations, i.e. the compositions, preparations or mixtures comprising the compound(s) (active ingredient(s)) of formula I and, where appropriate, a solid or liquid adjuvant, are prepared in known manner, e.g. by homogen eously mixing and/or grinding the active ingredient with extenders, e.g. solvents, solid carriers and, where appropriate, surface-active compounds (surfactants).

Further surfactants customarily used in formulation technology will be known to the person skilled in the art or can be found in the relevant technical literature.

The agrochemical compositions usually comprise 0.01 to 99% by weight, preferably 0.1 to 95% by weight, of a compound of formula I, 99.99 to 1% by weight, preferably 99.9 to 5% by weight, of a solid or liquid adjuvant, and 0 to 25% by weight, preferably 0.1 to 25% by weight, of a surfactant.

Whereas commercial products will preferably be formulated as concentrates, the end user will normally employ dilute formulations.

The compositions may also comprise further ingredients, such as stabilisers, antifoams, viscosity regulators, binders and tackifiers, as well as fertilisers or other active ingredients for obtaining special effects.

The Examples which follow illustrate the invention described above, without limiting the scope thereof in any way. Temperatures are given in degrees Celsius.

### PREPARATION EXAMPLES FOR COMPOUNDS OF FORMULA I

### Example A 1.1

(S)-N-{4-[3-(4-Chloro-phenyl)-prop-2-ynyloxy]-3-methoxy-benzyl}-2-ethanesulfonylamino-3-methyl-butyramide

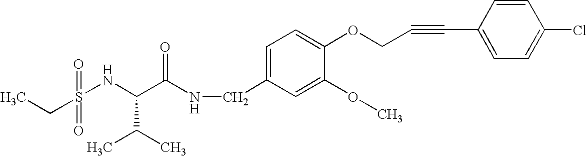

A mixture of (S)-2-ethanesulfonylamino-N-(4-hydroxy-3-methoxy-benzyl)-3-methyl-butyramide (1.5 g), toluene-4-sulfonic acid 3-(4-chloro-phenyl)-prop-2-ynyl ester (1.8 g) and sodium methoxide (7 ml, 1M solution in methanol) in methanol (30 ml) is heated to reflux for 2 hours. After cooling water (200 ml) is added. The mixture is extracted with ethyl acetate (2×300 ml). The organic layers are washed with brine (2×100 ml), combined, dried (MgSO₄) and the solvent is evaporated. (S)-N-{4-[3-(4-Chloro-phenyl)-prop-2-ynyloxy]-3-methoxy-benzyl}-2-ethanesulfonylamino-3-methyl-butyramide is obtained and purified by recrystallization (ethyl acetate/hexane), m.p. 162-163° C.

Analogously to example A1.1 the compounds listed in table A1 are obtained.

*) Configuration on the α-C-atom in the amino acid moiety; Ph means phenyl

TABLE A1

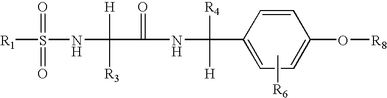

| No. | R₁ | *) | R₃ | R₄ | R₆ | R₈ | m.p. (° C.) |
|---|---|---|---|---|---|---|---|
| A1.1 | C₂H₅ | (S) | (CH₃)₂CH— | H | 3-OCH₃ | (4-Cl-Ph)-C≡C—CH₂— | 162-163 |
| A1.2 | C₂H₅ | (S) | (CH₃)₂CH— | H | 3-OCH₃ | (4-F-Ph)-C≡C—CH₂— | 138-139 |
| A1.3 | CH₃ | (S) | (CH₃)₂CH— | H | 3-OCH₃ | (4-Br-Ph)-C≡C—CH₂— | 144-147 |
| A1.4 | CH₃ | (S) | (CH₃)₂CH— | H | 3-OCH₃ | (4-CH₃-Ph)-C≡C—CH₂— | 160-162 |
| A1.5 | CH₃ | (S) | (CH₃)₂CH— | H | 3-OCH₃ | (4-Cl-Ph)-C≡C—CH₂— | 135-137 |
| A1.6 | CH₃ | (S) | (CH₃)₂CH— | H | 3-OCH₃ | (4-F-Ph)-C≡C—CH₂— | 125-127 |
| A1.7 | CH₃ | (S) | (CH₃)₂CH— | H | 3-OCH₃ | 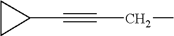 | 145-147 |
| A1.8 | CH₃ | (S) | (CH₃)₂CH— | H | 3-OCH₃ | (3,4-di-Cl-Ph)-CH₂— | 182-183 |
| A1.9 | CH₃ | (S) | (CH₃)₂CH— | H | 3-OCH₃ | (3-Cl-Ph)-CH₂— | 150-152 |
| A1.10 | CH₃ | (S) | (CH₃)₂CH— | H | 3-OCH₃ | (3-CF₃-Ph)-CH₂— | 163-164 |
| A1.11 | CH₃ | (S) | (CH₃)₂CH— | H | 3-OCH₃ | (4-CH₃-Ph)-(CH₂)₂—CH₂— | 165-167 |
| A1.12 | CH₃ | (S) | (CH₃)₂CH— | CH₃ | 3-OCH₃ | (4-Cl-Ph)-C≡C—CH₂— | 174-180 |
| A1.13 | CH₃ | (S) | (CH₃)₂CH— | H | 3-OCH₃ | (4-Cl-Ph)-CH₂—CH₂—CH₂— | 166-167 |
| A1.14 | CH₃— | (S) | (CH₃)₂CH— | H | 2-OCH₃ | (4-Cl-Ph)-C≡C—CH₂— | 182-184 |
| A1.15 | CH₃— | (S) | (CH₃)₂CH— | H | 2-OCH₃ | (4-Br-Ph)-C≡C—CH₂— | 186-188 |
| A1.16 | CH₃— | (S) | (CH₃)₂CH— | H | H | (4-Cl-Ph)-C≡C—CH₂— | 178-179 |
| A1.17 | CH₃— | (S) | (CH₃)₂CH— | H | H | (4-Br-Ph)-C≡C—CH₂— | 178-179 |
| A1.18 | CH₃— | (S) | (CH₃)₂CH— | H | 3-OC₂H₅ | (4-Cl-Ph)-C≡C—CH₂— | 166-167 |
| A1.19 | CH₃— | (S) | (CH₃)₂CH— | H | 3-OC₂H₅ | (4-Br-Ph)-C≡C—CH₂— | 169-170 |
| A1.20 | CH₃— | (S) | (CH₃)₂CH— | H | 3-Cl | (4-Cl-Ph)-C≡C—CH₂— | 144-147 |
| A1.21 | CH₃— | (S) | (CH₃)₂CH— | H | 3-Cl | (4-Br-Ph)-C≡C—CH₂— | 147-153 |
| A1.22 | CH₃— | (S) | (CH₃)₂CH— | H | 2-F | (4-Cl-Ph)-C≡C—CH₂— | 178-180 |
| A1.23 | CH₃— | (S) | (CH₃)₂CH— | H | 2-F | (4-Br-Ph)-C≡C—CH₂— | 189-190 |

### Example A2.1

(S)-2-Ethanesulfonylamino-N-(4-hydroxy-3-methoxy-benzyl)-3-methyl-butyramide

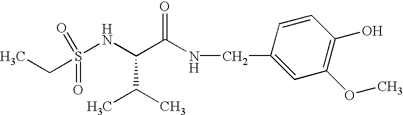

(S)-N-(4-Benzyloxy-3-methoxy-benzyl)-2-ethanesulfonylamino-3-methyl-butyramide (19.9 g) and palladium (12 g, 5% on charcoal) in tetrahydrofuran (200 ml) are shaken under a hydrogen atmosphere at +30 to +35° C. and at normal pressure for 3 hours. The reaction mixture is filtered and evaporated. (S)-2-Ethanesulfonylamino-N-(4-hydroxy-3-methoxy-benzyl)-3-methyl-butyramide is obtained, m.p. 49-50° C.

Analogously to example A2.1 the compounds listed in table A2 are obtained.

TABLE A2

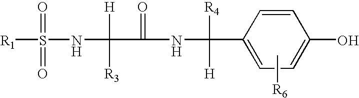

| No | R₁ | *) | R₃ | R₄ | R₆ | m.p. (° C.) |
|---|---|---|---|---|---|---|
| A2.1 | CH₃—CH₂— | (S) | (CH₃)₂CH— | H | 3-OCH₃ | 49-50 |
| A2.2 | CH₃— | (S) | (CH₃)₂CH— | H | 3-OCH₃ | 135-138 |
| A2.3 | CH₃— | (S) | (CH₃)₂CH— | CH₃ | 3-OCH₃ | 154-160 |
| A2.4 | CH₃— | (S) | (CH₃)₂CH— | H | H | 165-166 |
| A2.5 | CH₃— | (S) | (CH₃)₂CH— | H | 2-OCH₃ | 204-206 |
| A2.6 | CH₃— | (S) | (CH₃)₂CH— | H | 3-Cl | 160-161 |
| A2.7 | CH₃— | (S) | (CH₃)₂CH— | H | 3-OEt | 122-123 |
| A2.8 | CH₃— | (S) | (CH₃)₂CH— | H | 2-F | 188-190 |

*) Configuration of the amino acid moiety

### Example A3.1

(S)-N-(4-Benzyloxy-3-methoxy-benzyl)-2-ethanesulfonylamino-3-methyl-butyramide

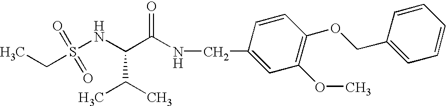

(S)-2-Ethanesulfonylamino-3-methyl-butyric acid (13.2 g), thionyl chloride (9 g) and N,N-dimethylformamide (2 drops) in toluene (70 ml) are refuxed for 3 hours. The solvent is then evaporated. To the resulting residue toluene (100 ml) is added. The solvent is then evaporated to dryness again. The residue is dissolved in dioxane (100 ml) and added to a mixture of 4-benzyloxy-3-methoxy-benzylamine (13.5 g) and triethylamine (5.9 g) in dioxane (100 ml). The reaction mixture is stirred at room temperature over night. Water (400 ml) is added. The mixture is extracted with ethyl acetate (2×500 ml). The organic layers are washed with hydrochloric acid (80 ml, 1M solution) and brine (2×100 ml), dried (MgSO₄) and evaporated. (S)-N-(4-Benzyloxy-3-methoxy-benzyl)-2-ethanesulfonylamino-3-methyl-butyramide which is purified by recrystallization (ethyl acetate/hexane), m.p. 141-152° C.

Analogously to example A3.1 the compounds listed in table A3 are obtained.

TABLE A3

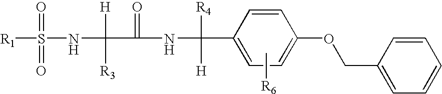

| | | | | | | m.p. |
|---|---|---|---|---|---|---|
| No | R₁ | *) | R₃ | R₄ | R₆ | (° C.) |
| A3.1 | CH₃—CH₂— | (S) | (CH₃)₂CH— | H | 3-OCH₃ | 141-152 |
| A3.2 | CH₃— | (S) | (CH₃)₂CH— | H | 3-OCH₃ | 156-158 |
| A3.3 | CH₃— | (S) | (CH₃)₂CH— | CH₃ | 3-OCH₃ | 142-170 |
| A3.4 | CH₃— | (S) | (CH₃)₂CH— | H | H | 133-134 |
| A3.5 | CH₃— | (S) | (CH₃)₂CH— | H | 2-OCH₃ | 156-157 |
| A3.6 | CH₃— | (S) | (CH₃)₂CH— | H | 3-Cl | 181-182 |
| A3.7 | CH₃— | (S) | (CH₃)₂CH— | H | 3-OC₂H₅ | 154-155 |
| A3.8 | CH₃— | (S) | (CH₃)₂CH— | H | 2-F | 147-148 |

*) Configuration of the amino acid moiety

Analogously to the above Examples the following compounds of Tables 1 to 27 may be prepared. In the tables Ph means phenyl.

TABLE 1
Compounds represented by the Formula I.1 wherein the combination of
the groups R₁, R₃ and R₈ corresponds to each row in table A.

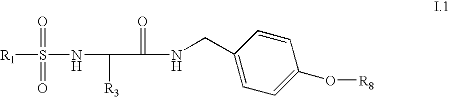

TABLE 2
Compounds represented by the Formula I.2 wherein the combination of
the groups R₁, R₃ and R₈ corresponds to each row in table A.

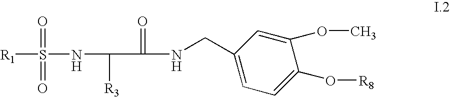

TABLE 3
Compounds represented by the Formula I.3 wherein the combination of
the groups R₁, R₃ and R₈ corresponds to each row in table A.

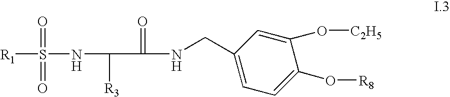

TABLE 4
Compounds represented by the Formula I.4 wherein the combination of
the groups R₁, R₃ and R₈ corresponds to each row in table A.

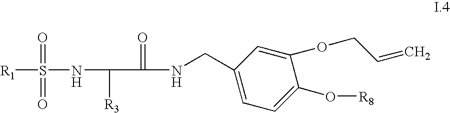

TABLE 5
Compounds represented by the Formula I.5 wherein the combination of
the groups R₁, R₃ and R₈ corresponds to each row in table A.

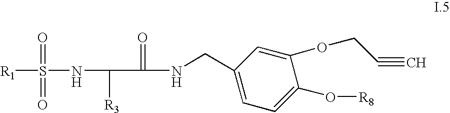

TABLE 6
Compounds represented by the Formula I.6 wherein the combination of
the groups R₁, R₃ and R₈ corresponds to each row in table A.

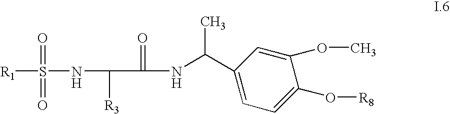

TABLE 7
Compounds represented by the Formula I.7 wherein the combination of
the groups R₁, R₃ and R₈ corresponds to each row in table A.

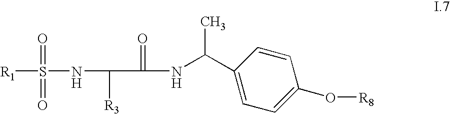

TABLE 8
Compounds represented by the Formula I.8 wherein the combination of
the groups R₁, R₃ and R₈ corresponds to each row in table A.

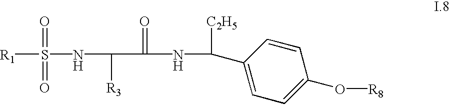

TABLE 9
Compounds represented by the Formula I.9 wherein the combination of
the groups R₁, R₃ and R₈ corresponds to each row in table A.

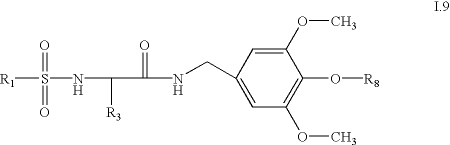

TABLE 10
Compounds represented by the Formula I.10 wherein the combination of
the groups R₁, R₃ and R₈ corresponds to each row in table A.

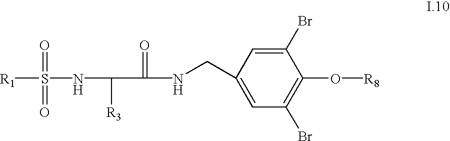

TABLE 11
Compounds represented by the Formula I.11 wherein the combination of
the groups R₁, R₃ and R₈ corresponds to each row in table A.

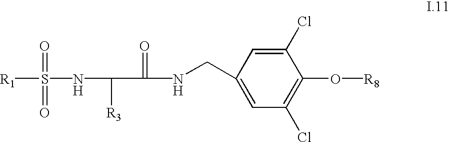

TABLE 12
Compounds represented by the Formula I.12 wherein the combination of
the groups R₁, R₃ and R₈ corresponds to each row in table A.

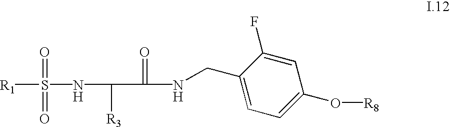

TABLE 13
Compounds represented by the Formula I.13 wherein the combination of
the groups R₁, R₃ and R₈ corresponds to each row in table A.

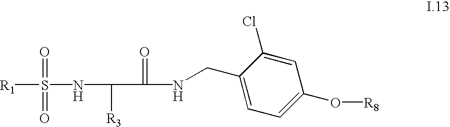

TABLE 14
Compounds represented by the Formula I.14 wherein the combination of
the groups R₁, R₃ and R₈ corresponds to each row in table A.

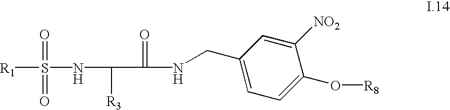

TABLE 15
Compounds represented by the Formula I.15 wherein the combination of
the groups R₁, R₃ and R₈ corresponds to each row in table A.

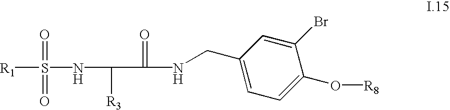

TABLE 16
Compounds represented by the Formula I.16 wherein the combination of
the groups R₁, R₃ and R₈ corresponds to each row in table A.

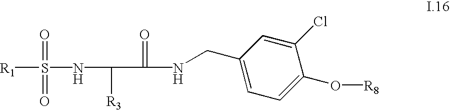

TABLE 17
Compounds represented by the Formula I.17 wherein the combination of
the groups R₁, R₃ and R₈ corresponds to each row in table A.

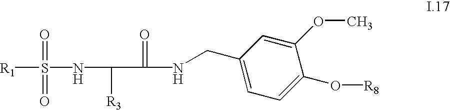

TABLE 18
Compounds represented by the Formula 1.18 wherein the combination of
the groups R₁, R₃ and R₈ corresponds to each row in table A.

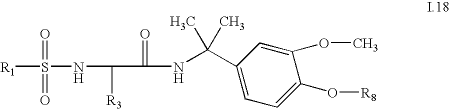

Table A

| No. | R₁ | R₃ | R₈ |
|---|---|---|---|
| 001 | CH₃— | CH₃—CH₂— | CH₃—(CH₂)₃—C≡C—CH₂— |
| 002 | CH₃—CH₂— | CH₃—CH₂— | CH₃—(CH₂)₃—C≡C—CH₂— |
| 003 | (CH₃)₂N— | CH₃—CH₂— | CH₃—(CH₂)₃—C≡C—CH₂— |
| 004 | CH₃—CH₂—CH₂— | CH₃—CH₂— | CH₃—(CH₂)₃—C≡C—CH₂— |
| 005 | (CH₃)₂CH— | CH₃—CH₂— | CH₃—(CH₂)₃—C≡C—CH₂— |
| 006 | Cl—CH₂—CH₂—CH₂— | CH₃—CH₂— | CH₃—(CH₂)₃—C≡C—CH₂— |
| 007 | CH₃— | (CH₃)₂CH— | CH₃—(CH₂)₃—C≡C—CH₂— |
| 008 | CH₃—CH₂— | (CH₃)₂CH— | CH₃—(CH₂)₃—C≡C—CH₂— |
| 009 | (CH₃)₂N— | (CH₃)₂CH— | CH₃—(CH₂)₃—C≡C—CH₂— |
| 010 | CH₃—CH₂—CH₂— | (CH₃)₂CH— | CH₃—(CH₂)₃—C≡C—CH₂— |
| 011 | (CH₃)₂CH— | (CH₃)₂CH— | CH₃—(CH₂)₃—C≡C—CH₂— |
| 012 | Cl—CH₂—CH₂—CH₂— | (CH₃)₂CH— | CH₃—(CH₂)₃—C≡C—CH₂— |
| 013 | CH₃— | CH₃—CH₂—CH₂— | CH₃—(CH₂)₃—C≡C—CH₂— |
| 014 | CH₃—CH₂— | CH₃—CH₂—CH₂— | CH₃—(CH₂)₃—C≡C—CH₂— |
| 015 | (CH₃)₂N— | CH₃—CH₂—CH₂— | CH₃—(CH₂)₃—C≡C—CH₂— |
| 016 | CH₃—CH₂—CH₂— | CH₃—CH₂—CH₂— | CH₃—(CH₂)₃—C≡C—CH₂— |
| 017 | (CH₃)₂CH— | CH₃—CH₂—CH₂— | CH₃(CH₂)₃—C≡C—CH₂— |
| 018 | Cl—CH₂—CH₂—CH₂— | CH₃—CH₂—CH₂— | CH₃—(CH₂)₃—C≡C—CH₂— |
| 019 | CH₃— | 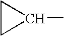 | CH₃—(CH₂)₃—C≡C—CH₂— |
| 020 | CH₃—CH₂— | 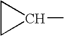 | CH₃—(CH₂)₃—C≡C—CH₂— |
| 021 | (CH₃)₂N— | 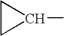 | CH₃—(CH₂)₃—C≡C—CH₂— |
| 022 | CH₃—CH₂—CH₂— | 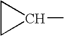 | CH₃—(CH₂)₃—C≡C—CH₂— |
| 023 | (CH₃)₂CH— | 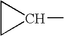 | CH₃—(CH₂)₃—C≡C—CH₂— |
| 024 | Cl—CH₂—CH₂—CH₂— | 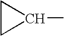 | CH₃—(CH₂)₃—C≡C—CH₂— |
| 025 | CH₃— | CH₂═CH—CH₂— | CH₃—(CH₂)₃—C≡C—CH₂— |
| 026 | CH₃—CH₂— | CH₂═CH—CH₂— | CH₃—(CH₂)₃—C≡C—CH₂— |
| 027 | (CH₃)₂N— | CH₂═CH—CH₂— | CH₃—(CH₂)₃—C≡C—CH₂— |
| 028 | CH₃—CH₂—CH₂— | CH₂═CH—CH₂— | CH₃—(CH₂)₃—C≡C—CH₂— |
| 029 | (CH₃)₂CH— | CH₂═CH—CH₂— | CH₃—(CH₂)₃—C≡C—CH₂— |
| 030 | Cl—CH₂—CH₂—CH₂— | CH₂═CH—CH₂— | CH₃—(CH₂)₃—C≡C—CH₂— |
| 031 | CH₃— | CH≡C—CH₂— | CH₃—(CH₂)₃—C≡C—CH₂— |
| 032 | CH₃—CH₂— | CH≡C—CH₂— | CH₃—(CH₂)₃—C≡C—CH₂— |
| 033 | (CH₃)₂N— | CH≡C—CH₂— | CH₃—(CH₂)₃—C≡C—CH₂— |
| 034 | CH₃—CH₂—CH₂— | CH≡C—CH₂— | CH₃—(CH₂)₃—C≡C—CH₂— |
| 035 | (CH₃)₂CH— | CH≡C—CH₂— | CH₃—(CH₂)₃—C≡C—CH₂— |
| 036 | Cl—CH₂—CH₂—CH₂— | CH≡C—CH₂— | CH₃—(CH₂)₃—C≡C—CH₂— |
| 037 | CH₃— | CH₃—CH₂—CH(CH₃)— | CH₃—(CH₂)₃—C≡C—CH₂— |
| 038 | CH₃—CH₂— | CH₃—CH₂—CH(CH₃)— | CH₃—(CH₂)₃—C≡C—CH₂— |
| 039 | (CH₃)₂N— | CH₃—CH₂—CH(CH₃)— | CH₃—(CH₂)₃—C≡C—CH₂— |
| 040 | CH₃—CH₂—CH₂— | CH₃—CH₂—CH(CH₃)— | CH₃—(CH₂)₃—C≡C—CH₂— |
| 041 | (CH₃)₂CH— | CH₃—CH₂—CH(CH₃)— | CH₃—(CH₂)₃—C≡C—CH₂— |
| 042 | Cl—CH₂—CH₂—CH₂— | CH₃—CH₂—CH(CH₃)— | CH₃—(CH₂)₃—C≡C—CH₂— |
| 045 | CH₃— | CH₃—CH₂— | 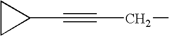 |
| 046 | CH₃—CH₂— | 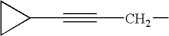 | |
| 047 | (CH₃)₂N— | CH₃—CH₂— | 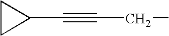 |
| 048 | CH₃—CH₂—CH₂— | CH₃—CH₂— | 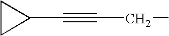 |
| 049 | (CH₃)₂CH— | CH₃—CH₂— | 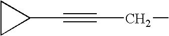 |
| 050 | Cl—CH₂—CH₂—CH₂— | CH₃—CH₂— | 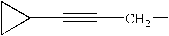 |
| 51 | CH₃— | (CH₃)₂CH— | 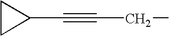 |
| 052 | CH₃—CH₂— | (CH₃)₂CH— | 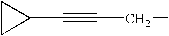 |
| 053 | (CH₃)₂N— | (CH₃)₂CH— | 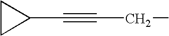 |
| 054 | CH₃—CH₂—CH₂— | (CH₃)₂CH— | 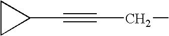 |
| 055 | (CH₃)₂CH— | (CH₃)₂CH— | 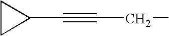 |
| 056 | Cl—CH₂—CH₂—CH₂— | (CH₃)₂CH— | 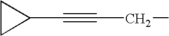 |
| 057 | CH₃— | CH₃—CH₂—CH₂— | 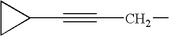 |
| 058 | CH₃—CH₂— | CH₃—CH₂—CH₂— | 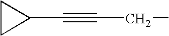 |
| 059 | (CH₃)₂N— | CH₃—CH₂—CH₂— | 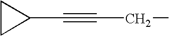 |
| 060 | CH₃—CH₂—CH₂— | CH₃—CH₂—CH₂— | 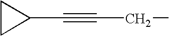 |
| 061 | (CH₃)₂CH— | CH₃—CH₂—CH₂— | 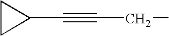 |
| 062 | Cl—CH₂—CH₂—CH₂— | CH₃—CH₂—CH₂— | 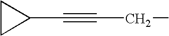 |
| 063 | CH₃— | 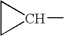 | 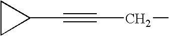 |
| 064 | CH₃—CH₂— | 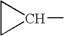 | 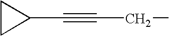 |
| 065 | (CH₃)₂N— | 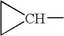 | 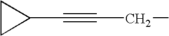 |
| 066 | CH₃—CH₂—CH₂— | 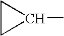 | 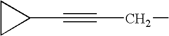 |
| 067 | (CH₃)₂CH— | 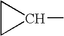 | 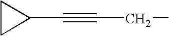 |
| 068 | Cl—CH₂—CH₂—CH₂— | 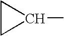 | 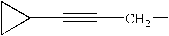 |
| 069 | CH₃— | CH₂═CH—CH₂— | 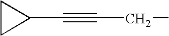 |
| 070 | CH₃—CH₂— | CH₂═CH—CH₂— | 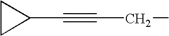 |
| 071 | (CH₃)₂N— | CH₂═CH—CH₂— | 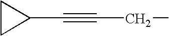 |
| 072 | CH₃—CH₂—CH₂— | CH₂═CH—CH₂— | 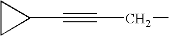 |
| 073 | (CH₃)₂CH— | CH₂═CH—CH₂— | 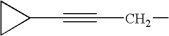 |
| 074 | Cl—CH₂—CH₂—CH₂— | CH₂═CH—CH₂— | 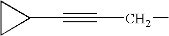 |
| 075 | CH₃— | CH≡C—CH₂— | 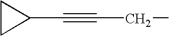 |
| 076 | CH₃—CH₂— | CH≡C—CH₂— | 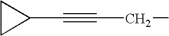 |
| 077 | (CH₃)₂N— | CH≡C—CH₂— | 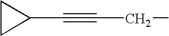 |
| 078 | CH₃—CH₂—CH₂— | CH≡C—CH₂— | 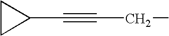 |
| 079 | (CH₃)₂CH— | CH≡C—CH₂— | 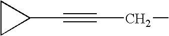 |
| 080 | Cl—CH₂—CH₂—CH₂— | CH≡C—CH₂— | 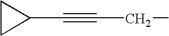 |
| 081 | CH₃— | CH₃—CH₂—CH(CH₃)— | 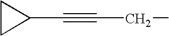 |
| 082 | CH₃—CH₂— | CH₃—CH₂—CH(CH₃)— | 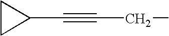 |
| 083 | (CH₃)₂N— | CH₃—CH₂—CH(CHHD 3)— | 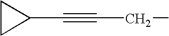 |
| 084 | CH₃—CH₂—CH₂— | CH₃—CH₂—CH(CH₃)— | 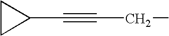 |
| 085 | (CH₃)₂CH— | CH₃—CH₂—CH(CH₃)— | 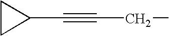 |
| 086 | Cl—CH₂—CH₂—CH₂— | CH₃—CH₂—CH(CH₃)— | 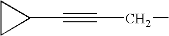 |
| 087 | CH₃— | CH₃—CH₂— | Ph—C≡C—CH₂— |
| 088 | CH₃—CH₂— | CH₃—CH₂— | Ph—C≡C—CH₂— |
| 089 | (CH₃)₂N— | CH₃—CH₂— | Ph—C≡C—CH₂— |
| 090 | CH₃—CH₂—CH₂— | CH₃—CH₂— | Ph—C≡C—CH₂— |
| 091 | (CH₃)₂CH— | CH₃—CH₂— | Ph—C≡C—CH₂— |
| 092 | Cl—CH₂—CH₂—CH₂— | CH₃—CH₂— | Ph—C≡C—CH₂— |
| 093 | CH₃— | (CH₃)₂CH— | Ph—C≡C—CH₂— |
| 094 | CH₃—CH₂— | (CH₃)₂CH— | Ph—C≡C—CH₂— |
| 095 | (CH₃)₂N— | (CH₃)₂CH— | Ph—C≡C—CH₂— |
| 096 | CH₃—CH₂—CH₂— | (CH₃)₂CH— | Ph—C≡C—CH₂— |
| 097 | (CH₃)₂CH— | (CH₃)₂CH— | Ph—C≡C—CH₂— |
| 098 | Cl—CH₂—CH₂—CH₂— | (CH₃)₂CH— | Ph—C≡C—CH₂— |
| 099 | CH₃— | CH₃—CH₂—CH₂— | Ph—C≡C—CH₂— |
| 100 | CH₃—CH₂— | CH₃—CH₂—CH₂— | Ph—C≡C—CH₂— |
| 101 | (CH₃)₂N— | CH₃—CH₂—CH₂— | Ph—C≡C—CH₂— |
| 102 | CH₃—CH₂—CH₂— | CH₃—CH₂—CH₂— | Ph—C≡C—CH₂— |
| 103 | (CH₃)₂CH— | CH₃—CH₂—CH₂— | Ph—C≡C—CH₂— |
| 104 | Cl—CH₂—CH₂—CH₂— | CH₃—CH₂—CH₂— | Ph—C≡C—CH₂— |
| 105 | CH₃— | 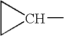 | Ph—C≡C—CH₂— |
| 106 | CH₃—CH₂— | 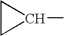 | Ph—C≡C—CH₂— |
| 107 | (CH₃)₂N— | 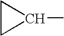 | Ph—C≡C—CH₂— |
| 108 | CH₃—CH₂—CH₂— | 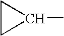 | Ph—C≡C—CH₂— |
| 109 | (CH₃)₂CH— | 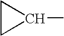 | Ph—C≡C—CH₂— |
| 110 | Cl—CH₂—CH₂—CH₂— | 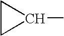 | Ph—C≡C—CH₂— |
| 111 | CH₃— | CH₂═CH—CH₂— | Ph—C≡C—CH₂— |
| 112 | CH₃—CH₂— | CH₂═CH—CH₂— | Ph—C≡C—CH₂— |
| 113 | (CH₃)₂N— | CH₂═CH—CH₂— | Ph—C≡C—CH₂— |
| 114 | CH₃—CH₂—CH₂— | CH₂═CH—CH₂— | Ph—C≡C—CH₂— |
| 115 | (CH₃)₂CH— | CH₂═CH—CH₂— | Ph—C≡C—CH₂— |
| 116 | Cl—CH₂—CH₂—CH₂— | CH₂═CH—CH₂— | Ph—C≡C—CH₂— |
| 117 | CH₃— | CH≡C—CH₂— | Ph—C≡C—CH₂— |
| 118 | CH₃—CH₂— | CH≡C—CH₂— | Ph—C≡C—CH₂— |
| 119 | (CH₃)₂N— | CH≡C—CH₂— | Ph—C≡C—CH₂— |
| 120 | CH₃—CH₂—CH₂— | CH≡C—CH₂— | Ph—C≡C—CH₂— |
| 121 | (CH₃)₂CH— | CH≡C—CH₂— | Ph—C≡C—CH₂— |
| 122 | Cl—CH₂—CH₂—CH₂— | CH≡C—CH₂— | Ph—C≡C—CH₂— |
| 123 | CH₃— | CH₃—CH₂—CH(CH₃)— | Ph—C≡C—CH₂— |
| 124 | CH₃—CH₂— | CH₃—CH₂—CH(CH₃)— | Ph—C≡C—CH₂— |
| 125 | (CH₃)₂N— | CH₃—CH₂—CH(CH₃)— | Ph—C≡C—CH₂— |
| 126 | CH₃—CH₂—CH₂— | CH₃—CH₂—CH(CH₃)— | Ph—C≡C—CH₂— |
| 127 | (CH₃)₂CH— | CH₃—CH₂—CH(CH₃)— | Ph—C≡C—CH₂— |
| 128 | Cl—CH₂—CH₂—CH₂— | CH₃—CH₂—CH(CH₃)— | Ph—C≡C—CH₂— |
| 129 | CH₃— | CH₃—CH₂— | (4-F—Ph)—C≡C—CH₂— |
| 130 | CH₃—CH₂— | CH₃—CH₂— | (4-F—Ph)—C≡C—CH₂— |
| 131 | (CH₃)₂N— | CH₃—CH₂— | (4-F—Ph)—C≡C—CH₂— |
| 132 | CH₃—CH₂—CH₂— | CH₃—CH₂— | (4-F—Ph)—C≡C—CH₂— |
| 133 | (CH₃)₂CH— | CH₃—CH₂— | (4-F—Ph)—C≡C—CH₂— |
| 134 | Cl—CH₂—CH₂—CH₂— | CH₃—CH₂— | (4-F—Ph)—C≡C—CH₂— |
| 135 | CH₃— | (CH₃)₂CH— | (4-F—Ph)—C≡C—CH₂— |
| 136 | CH₃—CH₂— | (CH₃)₂CH— | (4-F—Ph)—C≡C—CH₂— |
| 137 | (CH₃)₂N— | (CH₃)₂CH— | (4-F—Ph)—C≡C—CH₂— |
| 138 | CH₃—CH₂—CH₂— | (CH₃)₂CH— | (4-F—Ph)—C≡C—CH₂— |
| 139 | (CH₃)₂CH— | (CH₃)₂CH— | (4-F—Ph)—C≡C—CH₂— |
| 140 | Cl—CH₂—CH₂—CH₂— | (CH₃)₂CH— | (4-F—Ph)—C≡C—CH₂— |
| 141 | CH₃— | CH₃—CH₂—CH₂— | (4-F—Ph)—C≡C—CH₂— |
| 142 | CH₃—CH₂— | CH₃—CH₂—CH₂— | (4-F—Ph)—C≡C—CH₂— |
| 143 | (CH₃)₂N— | CH₃—CH₂—CH₂— | (4-F—Ph)—C≡C—CH₂— |
| 144 | CH₃—CH₂—CH₂— | CH₃—CH₂—CH₂— | (4-F—Ph)—C≡C—CH₂— |
| 145 | (CH₃)₂CH— | CH₃—CH₂—CH₂— | (4-F—Ph)—C≡C—CH₂— |
| 146 | Cl—CH₂—CH₂—CH₂— | CH₃—CH₂—CH₂— | (4-F—Ph)—C≡C—CH₂— |
| 147 | CH₃— | 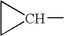 | (4-F—Ph)—C≡C—CH₂— |
| 148 | CH₃—CH₂— | 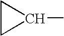 | (4-F—Ph)—C≡C—CH₂— |
| 149 | (CH₃)₂N— | 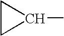 | (4-F—Ph)—C≡C—CH₂— |
| 150 | CH₃—CH₂—CH₂— | 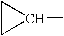 | (4-F—Ph)—C≡C—CH₂— |
| 151 | (CH₃)₂CH— | 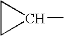 | (4-F—Ph)—C≡C—CH₂— |
| 152 | Cl—CH₂—CH₂—CH₂— | 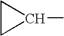 | (4-F—Ph)—C≡C—CH₂— |
| 153 | CH₃— | CH₂═CH—CH₂— | (4-F—Ph)—C≡C—CH₂— |
| 154 | CH₃—CH₂— | CH₂═CH—CH₂— | (4-F—Ph)—C≡C—CH₂— |
| 155 | (CH₃)₂N— | CH₂═CH—CH₂— | (4-F—Ph)—C≡C—CH₂— |
| 156 | CH₃—CH₂—CH₂— | CH₂═CH—CH₂— | (4-F—Ph)—C≡C—CH₂— |
| 157 | (CH₃)₂CH— | CH₂═CH—CH₂— | (4-F—Ph)—C≡C—CH₂— |
| 158 | Cl—CH₂—CH₂—CH₂— | CH₂═CH—CH₂— | (4-F—Ph)—C≡C—CH₂— |
| 159 | CH₃— | CH≡C—CH₂— | (4-F—Ph)—C≡C—CH₂— |
| 160 | CH₃—CH₂— | CH≡C—CH₂— | (4-F—Ph)—C≡C—CH₂— |
| 161 | (CH₃)₂N— | CH≡C—CH₂— | (4-F—Ph)—C≡C—CH₂— |
| 162 | CH₃—CH₂—CH₂— | CH≡C—CH₂— | (4-F—Ph)—C≡C—CH₂— |
| 163 | (CH₃)₂CH— | CH≡C—CH₂— | (4-F—Ph)—C≡C—CH₂— |
| 164 | Cl—CH₂—CH₂—CH₂— | CH≡C—CH₂— | (4-F—Ph)—C≡C—CH₂— |
| 165 | CH₃— | CH₃—CH₂—CH(CH₃)— | (4-F—Ph)—C≡C—CH₂— |
| 166 | CH₃—CH₂— | CH₃—CH₂—CH(CH₃)— | (4-F—Ph)—C≡C—CH₂— |
| 167 | (CH₃)₂N— | CH₃—CH₂—CH(CH₃)— | (4-F—Ph)—C≡C—CH₂— |
| 168 | CH₃—CH₂—CH₂— | CH₃—CH₂—CH(CH₃)— | (4-F—Ph)—C≡C—CH₂— |
| 169 | (CH₃)₂CH— | CH₃—CH₂—CH(CH₃)— | (4-F—Ph)—C≡C—CH₂— |
| 170 | Cl—CH₂—CH₂—CH₂— | CH₃—CH₂—CH(CH₃)— | (4-F—Ph)—C≡C—CH₂— |
| 171 | CH₃— | CH₃—CH₂— | (4-Cl—Ph)—C≡C—CH₂— |
| 172 | CH₃—CH₂— | CH₃—CH₂— | (4-Cl—Ph)—C≡C—CH₂— |
| 173 | (CH₃)₂N— | CH₃—CH₂— | (4-Cl—Ph)—C≡C—CH₂— |
| 174 | CH₃—CH₂—CH₂— | CH₃—CH₂— | (4-Cl—Ph)—C≡C—CH₂— |
| 175 | (CH₃)₂CH— | CH₃—CH₂— | (4-Cl—Ph)—C≡C—CH₂— |
| 176 | Cl—CH₂—CH₂—CH₂— | CH₃—CH₂— | (4-Cl—Ph)—C≡C—CH₂— |
| 177 | CH₃— | (CH₃)₂CH— | (4-Cl—Ph)—C≡C—CH₂— |
| 178 | CH₃—CH₂— | (CH₃)₂CH— | (4-Cl—Ph)—C≡C—CH₂— |
| 179 | (CH₃)₂N— | (CH₃)₂CH— | (4-Cl—Ph)—C≡C—CH₂— |
| 180 | CH₃—CH₂—CH₂— | (CH₃)₂CH— | (4-Cl—Ph)—C≡C—CH₂— |
| 181 | (CH₃)₂CH— | (CH₃)₂CH— | (4-Cl—Ph)—C≡C—CH₂— |
| 182 | Cl—CH₂—CH₂—CH₂— | (CH₃)₂CH— | (4-Cl—Ph)—C≡C—CH₂— |
| 183 | CH₃— | CH₃—CH₂—CH₂— | (4-Cl—Ph)—C≡C—CH₂— |
| 184 | CH₃—CH₂— | CH₃—CH₂—CH₂— | (4-Cl—Ph)—C≡C—CH₂— |
| 185 | (CH₃)₂N— | CH₃—CH₂—CH₂— | (4-Cl—Ph)—C≡C—CH₂— |
| 186 | CH₃—CH₂—CH₂— | CH₃—CH₂—CH₂— | (4-Cl—Ph)—C≡C—CH₂— |
| 187 | (CH₃)₂CH— | CH₃—CH₂—CH₂— | (4-Cl—Ph)—C≡C—CH₂— |
| 188 | Cl—CH₂—CH₂—CH₂— | CH₃—CH₂—CH₂— | (4-Cl—Ph)—C≡C—CH₂— |
| 189 | CH₃— | 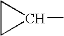 | (4-Cl—Ph)—C≡C—CH₂— |
| 190 | CH₃—CH₂— | 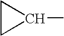 | (4-Cl—Ph)—C≡C—CH₂— |
| 191 | (CH₃)₂N— | 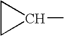 | (4-Cl—Ph)—C≡C—CH₂— |
| 192 | CH₃—CH₂—CH₂— | 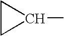 | (4-Cl—Ph)—C≡C—CH₂— |
| 193 | (CH₃)₂CH— | 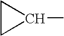 | (4-Cl—Ph)—C≡C—CH₂— |
| 194 | Cl—CH₂—CH₂—CH₂— | 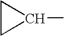 | (4-Cl—Ph)—C≡C—CH₂— |
| 195 | CH₃— | CH₂═CH—CH₂— | (4-Cl—Ph)—C≡C—CH₂— |
| 196 | CH₃—CH₂— | CH₂═CH—CH₂— | (4-Cl—Ph)—C≡C—CH₂— |
| 197 | (CH₃)₂N— | CH₂═CH—CH₂— | (4-Cl—Ph)—C≡C—CH₂— |
| 198 | CH₃—CH₂—CH₂— | CH₂═CH—CH₂— | (4-Cl—Ph)—C≡C—CH₂— |
| 199 | (CH₃)₂CH— | CH₂═CH—CH₂— | (4-Cl—Ph)—C≡C—CH₂— |
| 200 | Cl—CH₂—CH₂—CH₂— | CH₂═CH—CH₂— | (4-Cl—Ph)—C≡C—CH₂— |
| 201 | CH₃— | CH≡C—CH₂— | (4-Cl—Ph)—C≡C—CH₂— |
| 202 | CH₃—CH₂— | CH≡C—CH₂— | (4-Cl—Ph)—C≡C—CH₂— |
| 203 | (CH₃)₂N— | CH≡C—CH₂— | (4-Cl—Ph)—C≡C—CH₂— |
| 204 | CH₃—CH₂—CH₂— | CH≡C—CH₂— | (4-Cl—Ph)—C≡C—CH₂— |
| 205 | (CH₃)₂CH— | CH≡C—CH₂— | (4-Cl—Ph)—C≡C—CH₂— |
| 206 | Cl—CH₂—CH₂—CH₂— | CH≡C—CH₂— | (4-Cl—Ph)—C≡C—CH₂— |
| 207 | CH₃— | CH₃—CH₂—CH(CH₃)— | (4-Cl—Ph)—C≡C—CH₂— |
| 208 | CH₃—CH₂— | CH₃—CH₂—CH(CH₃)— | (4-Cl—Ph)—C≡C—CH₂— |
| 209 | (CH₃)₂N— | CH₃—CH₂—CH(CH₃)— | (4-Cl—Ph)—C≡C—CH₂— |
| 210 | CH₃—CH₂—CH₂— | CH₃—CH₂—CH(CH₃)— | (4-Cl—Ph)—C≡C—CH₂— |
| 211 | (CH₃)₂CH— | CH₃—CH₂—CH(CH₃)— | (4-Cl—Ph)—C≡C—CH₂— |
| 212 | Cl—CH₂—CH₂—CH₂— | CH₃—CH₂—CH(CH₃)— | (4-Cl—Ph)—C≡C—CH₂— |
| 213 | CH₃— | CH₃—CH₂— | (4-Br—Ph)—C≡C—CH₂— |
| 214 | CH₃—CH₂— | CH₃—CH₂— | (4-Br—Ph)—C≡C—CH₂— |
| 215 | (CH₃)₂N— | CH₃—CH₂— | (4-Br—Ph)—C≡C—CH₂— |
| 216 | CH₃—CH₂—CH₂— | CH₃—CH₂— | (4-Br—Ph)—C≡C—CH₂— |
| 217 | (CH₃)₂CH— | CH₃—CH₂— | (4-Br—Ph)—C≡C—CH₂— |
| 218 | Cl—CH₂—CH₂—CH₂— | CH₃—CH₂— | (4-Br—Ph)—C≡C—CH₂— |
| 219 | CH₃— | (CH₃)₂CH— | (4-Br—Ph)—C≡C—CH₂— |
| 220 | CH₃—CH₂— | (CH₃)₂CH— | (4-Br—Ph)—C≡C—CH₂— |
| 221 | (CH₃)₂N— | (CH₃)₂CH— | (4-Br—Ph)—C≡C—CH₂— |
| 222 | CH₃—CH₂—CH₂— | (CH₃)₂CH— | (4-Br—Ph)—C≡C—CH₂— |
| 223 | (CH₃)₂CH— | (CH₃)₂CH— | (4-Br—Ph)—C≡C—CH₂— |
| 224 | Cl—CH₂—CH₂—CH₂— | (CH₃)₂CH— | (4-Br—Ph)—C≡C—CH₂— |
| 225 | CH₃— | CH₃—CH₂—CH₂— | (4-Br—Ph)—C≡C—CH₂— |
| 226 | CH₃—CH₂— | CH₃—CH₂—CH₂— | (4-Br—Ph)—C≡C—CH₂— |
| 227 | (CH₃)₂N— | CH₃—CH₂—CH₂— | (4-Br—Ph)—C≡C—CH₂— |
| 228 | CH₃—CH₂—CH₂— | CH₃—CH₂—CH₂— | (4-Br—Ph)—C≡C—CH₂— |
| 229 | (CH₃)₂CH— | CH₃—CH₂—CH₂— | (4-Br—Ph)—C≡C—CH₂— |
| 230 | Cl—CH₂—CH₂—CH₂— | CH₃—CH₂—CH₂— | (4-Br—Ph)—C≡C—CH₂— |
| 231 | CH₃— | 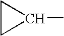 | (4-Br—Ph)—C≡C—CH₂— |
| 232 | CH₃—CH₂— | 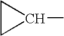 | (4-Br—Ph)—C≡C—CH₂— |
| 233 | (CH₃)₂N— | 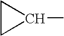 | (4-Br—Ph)—C≡C—CH₂— |
| 234 | CH₃—CH₂—CH₂— | 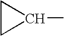 | (4-Br—Ph)—C≡C—CH₂— |
| 235 | (CH₃)₂CH— | 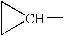 | (4-Br—Ph)—C≡C—CH₂— |
| 236 | Cl—CH₂—CH₂—CH₂— | 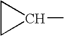 | (4-Br—Ph)—C≡C—CH₂— |
| 237 | CH₃— | CH₂═CH—CH₂— | (4-Br—Ph)—C≡C—CH₂— |
| 238 | CH₃—CH₂— | CH₂═CH—CH₂— | (4-Br—Ph)—C≡C—CH₂— |
| 239 | (CH₃)₂N— | CH₂═CH—CH₂— | (4-Br—Ph)—C≡C—CH₂— |
| 240 | CH₃—CH₂—CH₂— | CH₂═CH—CH₂— | (4-Br—Ph)—C≡C—CH₂— |
| 241 | (CH₃)₂CH— | CH₂═CH—CH₂— | (4-Br—Ph)—C≡C—CH₂— |
| 242 | Cl—CH₂—CH₂—CH₂— | CH₂═CH—CH₂— | (4-Br—Ph)—C≡C—CH₂— |
| 243 | CH₃— | CH≡C—CH₂— | (4-Br—Ph)—C≡C—CH₂— |
| 244 | CH₃—CH₂— | CH≡C—CH₂— | (4-Br—Ph)—C≡C—CH₂— |
| 245 | (CH₃)₂N— | CH≡C—CH₂— | (4-Br—Ph)—C≡C—CH₂— |
| 246 | CH₃—CH₂—CH₂— | CH≡C—CH₂— | (4-Br—Ph)—C≡C—CH₂— |
| 247 | (CH₃)₂CH— | CH≡C—CH₂— | (4-Br—Ph)—C≡C—CH₂— |
| 248 | Cl—CH₂—CH₂—CH₂— | CH≡C—CH₂— | (4-Br—Ph)—C≡C—CH₂— |
| 249 | CH₃— | CH₃—CH₂—CH(CH₃)— | (4-Br—Ph)—C≡C—CH₂— |
| 250 | CH₃—CH₂— | CH₃—CH₂—CH(CH₃)— | (4-Br—Ph)—C≡C—CH₂— |
| 251 | (CH₃)₂N— | CH₃—CH₂—CH(CH₃)— | (4-Br—Ph)—C≡C—CH₂— |
| 252 | CH₃—CH₂—CH₂— | CH₃—CH₂—CH(CH₃)— | (4-Br—Ph)—C≡C—CH₂— |
| 253 | (CH₃)₂CH— | CH₃—CH₂—CH(CH₃)— | (4-Br—Ph)—C≡C—CH₂— |
| 254 | Cl—CH₂—CH₂—CH₂— | CH₃—CH₂—CH(CH₃)— | (4-Br—Ph)—C≡C—CH₂— |
| 255 | CH₃— | CH₃—CH₂— | H |
| 256 | CH₃—CH₂— | CH₃—CH₂— | H |
| 257 | (CH₃)₂N— | CH₃—CH₂— | H |
| 258 | CH₃—CH₂—CH₂— | CH₃—CH₂— | H |
| 259 | (CH₃)₂CH— | CH₃—CH₂— | H |
| 260 | Cl—CH₂—CH₂—CH₂— | CH₃—CH₂— | H |
| 261 | CH₃— | (CH₃)₂CH— | H |
| 262 | CH₃—CH₂— | (CH₃)₂CH— | H |
| 263 | (CH₃)₂N— | (CH₃)₂CH— | H |
| 264 | CH₃—CH₂—CH₂— | (CH₃)₂CH— | H |
| 265 | (CH₃)₂CH— | (CH₃)₂CH— | H |
| 266 | Cl—CH₂—CH₂—CH₂— | (CH₃)₂CH— | H |
| 267 | CH₃— | CH₃—CH₂—CH₂— | H |
| 268 | CH₃—CH₂— | CH₃—CH₂—CH₂— | H |
| 269 | (CH₃)₂N— | CH₃—CH₂—CH₂— | H |
| 270 | CH₃—CH₂—CH₂— | CH₃—CH₂—CH₂— | H |
| 271 | (CH₃)₂CH— | CH₃—CH₂—CH₂— | H |
| 272 | Cl—CH₂—CH₂—CH₂— | CH₃—CH₂—CH₂— | H |
| 273 | CH₃— | 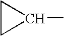 | H |
| 274 | CH₃—CH₂— | 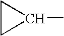 | H |
| 275 | (CH₃)₂N— | 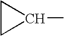 | H |
| 276 | CH₃—CH₂—CH₂— | 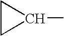 | H |
| 277 | (CH₃)₂CH— | 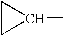 | H |
| 278 | Cl—CH₂—CH₂—CH₂— | 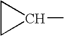 | H |
| 279 | CH₃— | CH₂═CH—CH₂— | H |
| 280 | CH₃—CH₂— | CH₂═CH—CH₂— | H |
| 281 | (CH₃)₂N— | CH₂═CH—CH₂— | H |
| 282 | CH₃—CH₂—CH₂— | CH₂═CH—CH₂— | H |
| 283 | (CH₃)₂CH— | CH₂═CH—CH₂— | H |
| 284 | Cl—CH₂—CH₂—CH₂— | CH₂═CH—CH₂— | H |
| 285 | CH₃— | CH≡C—CH₂— | H |
| 286 | CH₃—CH₂— | CH≡C—CH₂— | H |
| 287 | (CH₃)₂N— | CH≡C—CH₂— | H |
| 288 | CH₃—CH₂—CH₂— | CH≡C—CH₂— | H |
| 289 | (CH₃)₂CH— | CH≡C—CH₂— | H |
| 290 | Cl—CH₂—CH₂—CH₂— | CH≡C—CH₂— | H |
| 291 | CH₃— | CH₃—CH₂—CH(CH₃)— | H |
| 292 | CH₃—CH₂— | CH₃—CH₂—CH(CH₃)— | H |
| 293 | (CH₃)₂N— | CH₃—CH₂—CH(CH₃)— | H |
| 294 | CH₃—CH₂—CH₂— | CH₃—CH₂—CH(CH₃)— | H |
| 295 | (CH₃)₂CH— | CH₃—CH₂—CH(CH₃)— | H |
| 296 | Cl—CH₂—CH₂—CH₂— | CH₃—CH₂—CH(CH₃)— | H |
| 297 | CH₃— | CH₃—CH₂— | Ph—CH₂— |
| 298 | CH₃—CH₂— | CH₃—CH₂— | Ph—CH₂— |
| 299 | (CH₃)₂N— | CH₃—CH₂— | Ph—CH₂— |
| 300 | CH₃—CH₂—CH₂— | CH₃—CH₂— | Ph—CH₂— |
| 301 | (CH₃)₂CH— | CH₃—CH₂— | Ph—CH₂— |
| 302 | Cl—CH₂—CH₂—CH₂— | CH₃—CH₂— | Ph—CH₂— |
| 303 | CH₃— | (CH₃)₂CH— | Ph—CH₂— |
| 304 | CH₃—CH₂— | (CH₃)₂CH— | Ph—CH₂— |
| 305 | (CH₃)₂N— | (CH₃)₂CH— | Ph—CH₂— |
| 306 | CH₃—CH₂—CH₂— | (CH₃)₂CH— | Ph—CH₂— |
| 307 | (CH₃)₂CH— | (CH₃)₂CH— | Ph—CH₂— |
| 308 | Cl—CH₂—CH₂—CH₂— | (CH₃)₂CH— | Ph—CH₂— |
| 309 | CH₃— | CH₃—CH₂—CH₂— | Ph—CH₂— |
| 310 | CH₃—CH₂— | CH₃—CH₂—CH₂— | Ph—CH₂— |
| 311 | (CH₃)₂N— | CH₃—CH₂—CH₂— | Ph—CH₂— |
| 312 | CH₃—CH₂—CH₂— | CH₃—CH₂—CH₂— | Ph—CH₂— |
| 313 | (CH₃)₂CH— | CH₃—CH₂—CH₂— | Ph—CH₂— |
| 314 | Cl—CH₂—CH₂—CH₂— | CH₃—CH₂—CH₂— | Ph—CH₂— |
| 315 | CH₃— | 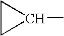 | Ph—CH₂— |
| 316 | CH₃—CH₂— | 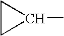 | Ph—CH₂— |
| 317 | (CH₃)₂N— | 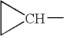 | Ph—CH₂— |
| 318 | CH₃—CH₂—CH₂— | 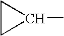 | Ph—CH₂— |
| 319 | (CH₃)₂CH— | 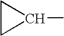 | Ph—CH₂— |
| 320 | Cl—CH₂—CH₂—CH₂— | 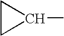 | Ph—CH₂— |
| 321 | CH₃— | CH₂═CH—CH₂— | Ph—CH₂— |
| 322 | CH₃—CH₂— | CH₂═CH—CH₂— | Ph—CH₂— |
| 323 | (CH₃)₂N— | CH₂═CH—CH₂— | Ph—CH₂— |
| 324 | CH₃—CH₂—CH₂— | CH₂═CH—CH₂— | Ph—CH₂— |
| 325 | (CH₃)₂CH— | CH₂═CH—CH₂— | Ph—CH₂— |
| 326 | Cl—CH₂—CH₂—CH₂— | CH₂═CH—CH₂— | Ph—CH₂— |
| 327 | CH₃— | CH≡C—CH₂— | Ph—CH₂— |
| 328 | CH₃—CH₂— | CH≡C—CH₂— | Ph—CH₂— |
| 329 | (CH₃)₂N— | CH≡C—CH₂— | Ph—CH₂— |
| 330 | CH₃—CH₂—CH₂— | CH≡C—CH₂— | Ph—CH₂— |
| 331 | (CH₃)₂CH— | CH≡C—CH₂— | Ph—CH₂— |
| 332 | Cl—CH₂—CH₂—CH₂— | CH≡C—CH₂— | Ph—CH₂— |
| 333 | CH₃— | CH₃—CH₂—CH(CH₃)— | Ph—CH₂— |
| 334 | CH₃—CH₂— | CH₃—CH₂—CH(CH₃)— | Ph—CH₂— |
| 335 | (CH₃)₂N— | CH₃—CH₂—CH(CH₃)— | Ph—CH₂— |
| 336 | CH₃—CH₂—CH₂— | CH₃—CH₂—CH(CH₃)— | Ph—CH₂— |
| 337 | (CH₃)₂CH— | CH₃—CH₂—CH(CH₃)— | Ph—CH₂— |
| 338 | Cl—CH₂—CH₂—CH₂— | CH₃—CH₂—CH(CH₃)— | Ph—CH₂— |
| 339 | CH₃— | CH₃—CH₂— | (4-Cl—Ph)—CH₂— |
| 340 | CH₃—CH₂— | CH₃—CH₂— | (4-Cl—Ph)—CH₂— |
| 341 | (CH₃)₂N— | CH₃—CH₂— | (4-Cl—Ph)—CH₂— |
| 342 | CH₃—CH₂—CH₂— | CH₃—CH₂— | (4-Cl—Ph)—CH₂— |
| 343 | (CH₃)₂CH— | CH₃—CH₂— | (4-Cl—Ph)—CH₂— |
| 344 | Cl—CH₂—CH₂—CH₂— | CH₃—CH₂— | (4-Cl—Ph)—CH₂— |
| 345 | CH₃— | (CH₃)₂CH— | (4-Cl—Ph)—CH₂— |
| 346 | CH₃—CH₂— | (CH₃)₂CH— | (4-Cl—Ph)—CH₂— |
| 347 | (CH₃)₂N— | (CH₃)₂CH— | (4-Cl—Ph)—CH₂— |
| 348 | CH₃—CH₂—CH₂— | (CH₃)₂CH— | (4-Cl—Ph)—CH₂— |
| 349 | (CH₃)₂CH— | (CH₃)₂CH— | (4-Cl—Ph)—CH₂— |
| 350 | Cl—CH₂—CH₂—CH₂— | (CH₃)₂CH— | (4-Cl—Ph)—CH₂— |
| 351 | CH₃— | CH₃—CH₂—CH₂— | (4-Cl—Ph)—CH₂— |
| 352 | CH₃—CH₂— | CH₃—CH₂—CH₂— | (4-Cl—Ph)—CH₂— |
| 353 | (CH₃)₂N— | CH₃—CH₂—CH₂— | (4-Cl—Ph)—CH₂— |
| 354 | CH₃—CH₂—CH₂— | CH₃—CH₂—CH₂— | (4-Cl—Ph)—CH₂— |
| 355 | (CH₃)₂CH— | CH₃—CH₂—CH₂— | (4-Cl—Ph)—CH₂— |
| 356 | Cl—CH₂—CH₂—CH₂— | CH₃—CH₂—CH₂— | (4-Cl—Ph)—CH₂— |
| 357 | CH₃— | 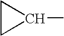 | (4-Cl—Ph)—CH₂— |
| 358 | CH₃—CH₂— | 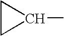 | (4-Cl—Ph)—CH₂— |
| 359 | (CH₃)₂N— | 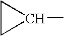 | (4-Cl—Ph)—CH₂— |
| 360 | CH₃—CH₂—CH₂— | 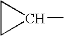 | (4-Cl—Ph)—CH₂— |
| 361 | (CH₃)₂CH— | 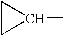 | (4-Cl—Ph)—CH₂— |
| 362 | Cl—CH₂—CH₂—CH₂— | 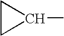 | (4-Cl—Ph)—CH₂— |
| 363 | CH₃— | CH₂═CH—CH₂— | (4-Cl—Ph)—CH₂— |
| 364 | CH₃—CH₂— | CH₂═CH—CH₂— | (4-Cl—Ph)—CH₂— |
| 365 | (CH₃)₂N— | CH₂═CH—CH₂— | (4-Cl—Ph)—CH₂— |
| 366 | CH₃—CH₂—CH₂— | CH₂═CH—CH₂— | (4-Cl—Ph)—CH₂— |
| 367 | (CH₃)₂CH— | CH₂═CH—CH₂— | (4-Cl—Ph)—CH₂— |
| 368 | Cl—CH₂—CH₂—CH₂— | CH₂═CH—CH₂— | (4-Cl—Ph)—CH₂— |
| 369 | CH₃— | CH≡C—CH₂— | (4-Cl—Ph)—CH₂— |
| 370 | CH₃—CH₂— | CH≡C—CH₂— | (4-Cl—Ph)—CH₂— |
| 371 | (CH₃)₂N— | CH≡C—CH₂— | (4-Cl—Ph)—CH₂— |
| 372 | CH₃—CH₂—CH₂— | CH≡C—CH₂— | (4-Cl—Ph)—CH₂— |
| 373 | (CH₃)₂CH— | CH≡C—CH₂— | (4-Cl—Ph)—CH₂— |
| 374 | Cl—CH₂—CH₂—CH₂— | CH≡C—CH₂— | (4-Cl—Ph)—CH₂— |
| 375 | CH₃— | CH₃—CH₂—CH(CH₃)— | (4-Cl—Ph)—CH₂— |
| 376 | CH₃—CH₂— | CH₃—CH₂—CH(CH₃)— | (4-Cl—Ph)—CH₂— |
| 378 | (CH₃)₂N— | CH₃—CH₂—CH(CH₃)— | (4-Cl—Ph)—CH₂— |
| 379 | CH₃—CH₂—CH₂— | CH₃—CH₂—CH(CH₃)— | (4-Cl—Ph)—CH₂— |
| 380 | (CH₃)₂CH— | CH₃—CH₂—CH(CH₃)— | (4-Cl—Ph)—CH₂— |
| 381 | Cl—CH₂—CH₂—CH₂— | CH₃—CH₂—CH(CH₃)— | (4-Cl—Ph)—CH₂— |
| 382 | CH₃— | CH₃—CH₂— | (3-Cl—Ph)—CH₂— |
| 383 | CH₃—CH₂— | CH₃—CH₂— | (3-Cl—Ph)—CH₂— |
| 384 | (CH₃)₂N— | CH₃—CH₂— | (3-Cl—Ph)—CH₂— |
| 385 | CH₃—CH₂—CH₂— | CH₃—CH₂— | (3-Cl—Ph)—CH₂— |
| 386 | (CH₃)₂CH— | CH₃—CH₂— | (3-Cl—Ph)—CH₂— |
| 387 | Cl—CH₂—CH₂—CH₂— | CH₃—CH₂— | (3-Cl—Ph)—CH₂— |
| 388 | CH₃— | (CH₃)₂CH— | (3-Cl—Ph)—CH₂— |
| 389 | CH₃—CH₂— | (CH₃)₂CH— | (3-Cl—Ph)—CH₂— |
| 390 | (CH₃)₂N— | (CH₃)₂CH— | (3-Cl—Ph)—CH₂— |
| 391 | CH₃—CH₂—CH₂— | (CH₃)₂CH— | (3-Cl—Ph)—CH₂— |
| 392 | (CH₃)₂CH— | (CH₃)₂CH— | (3-Cl—Ph)—CH₂— |
| 393 | Cl—CH₂—CH₂—CH₂— | (CH₃)₂CH— | (3-Cl—Ph)—CH₂— |
| 394 | CH₃— | CH₃—CH₂—CH₂— | (3-Cl—Ph)—CH₂— |
| 395 | CH₃—CH₂— | CH₃—CH₂—CH₂— | (3-Cl—Ph)—CH₂— |
| 396 | (CH₃)₂N— | CH₃—CH₂—CH₂— | (3-Cl—Ph)—CH₂— |
| 397 | CH₃—CH₂—CH₂— | CH₃—CH₂—CH₂— | (3-Cl—Ph)—CH₂— |
| 398 | (CH₃)₂CH— | CH₃—CH₂—CH₂— | (3-Cl—Ph)—CH₂— |
| 399 | Cl—CH₂—CH₂—CH₂— | CH₃—CH₂—CH₂— | (3-Cl—Ph)—CH₂— |
| 400 | CH₃— | 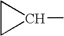 | (3-Cl—Ph)—CH₂— |
| 401 | CH₃—CH₂— | 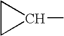 | (3-Cl—Ph)—CH₂— |
| 402 | (CH₃)₂N— | 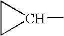 | (3-Cl—Ph)—CH₂— |
| 403 | CH₃—CH₂—CH₂— | 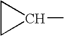 | (3-Cl—Ph)—CH₂— |
| 404 | (CH₃)₂CH— | 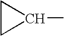 | (3-Cl—Ph)—CH₂— |
| 405 | Cl—CH₂—CH₂—CH₂— | 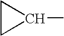 | (3-Cl—Ph)—CH₂— |
| 406 | CH₃— | CH₂═CH—CH₂— | (3-Cl—Ph)—CH₂— |
| 407 | CH₃—CH₂— | CH₂═CH—CH₂— | (3-Cl—Ph)—CH₂— |
| 408 | (CH₃)₂N— | CH₂═CH—CH₂— | (3-Cl—Ph)—CH₂— |
| 409 | CH₃—CH₂—CH₂— | CH₂═CH—CH₂— | (3-Cl—Ph)—CH₂— |
| 410 | (CH₃)₂CH— | CH₂═CH—CH₂— | (3-Cl—Ph)—CH₂— |
| 411 | Cl—CH₂—CH₂—CH₂— | CH₂═CH—CH₂— | (3-Cl—Ph)—CH₂— |
| 412 | CH₃— | CH≡C—CH₂— | (3-Cl—Ph)—CH₂— |
| 413 | CH₃—CH₂— | CH≡C—CH₂— | (3-Cl—Ph)—CH₂— |
| 414 | (CH₃)₂N— | CH≡C—CH₂— | (3-Cl—Ph)—CH₂— |
| 415 | CH₃—CH₂—CH₂— | CH≡C—CH₂— | (3-Cl—Ph)—CH₂— |
| 416 | (CH₃)₂CH— | CH≡C—CH₂— | (3-Cl—Ph)—CH₂— |
| 417 | Cl—CH₂—CH₂—CH₂— | CH≡C—CH₂— | (3-Cl—Ph)—CH₂— |
| 418 | CH₃— | CH₃—CH₂—CH(CH₃)— | (3-Cl—Ph)—CH₂— |
| 419 | CH₃—CH₂— | CH₃—CH₂—CH(CH₃)— | (3-Cl—Ph)—CH₂— |
| 420 | (CH₃)₂N— | CH₃—CH₂—CH(CH₃)— | (3-Cl—Ph)—CH₂— |
| 421 | CH₃—CH₂—CH₂— | CH₃—CH₂—CH(CH₃)— | (3-Cl—Ph)—CH₂— |
| 422 | (CH₃)₂CH— | CH₃—CH₂—CH(CH₃)— | (3-Cl—Ph)—CH₂— |
| 423 | Cl—CH₂—CH₂—CH₂— | CH₃—CH₂—CH(CH₃)— | (3-Cl—Ph)—CH₂— |
| 424 | CH₃— | CH₃—CH₂— | (3-CF₃—Ph)—CH₂— |
| 425 | CH₃—CH₂— | CH₃—CH₂— | (3—CF₃—Ph)—CH₂— |
| 426 | (CH₃)₂N— | CH₃—CH₂— | (3—CF₃—Ph)—CH₂— |
| 427 | CH₃—CH₂—CH₂— | CH₃—CH₂— | (3-CF₃—Ph)—CH₂— |
| 428 | (CH₃)₂CH— | CH₃—CH₂— | (3-CF₃—Ph)—CH₂— |
| 429 | Cl—CH₂—CH₂—CH₂— | CH₃—CH₂— | (3-CF₃—Ph)—CH₂— |
| 430 | CH₃— | (CH₃)₂CH— | (3-CF₃—Ph)—CH₂— |
| 431 | CH₃—CH₂— | (CH₃)₂CH— | (3-CF₃—Ph)—CH₂— |
| 432 | (CH₃)₂N— | (CH₃)₂CH— | (3-CF₃—Ph)—CH₂— |
| 433 | CH₃—CH₂—CH₂— | (CH₃)₂CH— | (3-CF₃—Ph)—CH₂— |
| 434 | (CH₃)₂CH— | (CH₃)₂CH— | (3-CF₃—Ph)—CH₂— |
| 435 | Cl—CH₂—CH₂—CH₂— | (CH₃)₂CH— | (3-CF₃—Ph)—CH₂— |
| 436 | CH₃— | CH₃—CH₂—CH₂— | (3-CF₃—Ph)—CH₂— |
| 437 | CH₃—CH₂— | CH₃—CH₂—CH₂— | (3-CF₃—Ph)—CH₂— |
| 438 | (CH₃)₂N— | CH₃—CH₂—CH₂— | (3-CF₃—Ph)—CH₂— |
| 439 | CH₃—CH₂—CH₂— | CH₃—CH₂—CH₂— | (3-CF₃—Ph)—CH₂— |
| 440 | (CH₃)₂CH— | CH₃—CH₂—CH₂— | (3-CF₃—Ph)—CH₂— |
| 441 | Cl—CH₂—CH₂—CH₂— | CH₃—CH₂—CH₂— | (3-CF₃—Ph)—CH₂— |
| 442 | CH₃— | 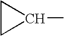 | (3-CF₃—Ph)—CH₂— |
| 443 | CH₃—CH₂— | 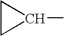 | (3-CF₃—Ph)—CH₂— |
| 444 | (CH₃)₂N— | 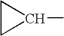 | (3-CF₃—Ph)—CH₂— |
| 445 | CH₃—CH₂—CH₂— | 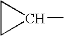 | (3-CF₃—Ph)—CH₂— |
| 446 | (CH₃)₂CH— | 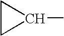 | (3-CF₃—Ph)—CH₂— |
| 447 | Cl—CH₂—CH₂—CH₂— | 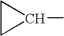 | (3-CF₃—Ph)—CH₂— |
| 448 | CH₃— | CH₂═CH—CH₂— | (3-CF₃—Ph)—CH₂— |
| 449 | CH₃—CH₂— | CH₂═CH—CH₂— | (3-CF₃—Ph)—CH₂— |
| 450 | (CH₃)₂N— | CH₂═CH—CH₂— | (3-CF₃—Ph)—CH₂— |
| 451 | CH₃—CH₂—CH₂— | CH₂═CH—CH₂— | (3-CF₃—Ph)—CH₂— |
| 452 | (CH₃)₂CH— | CH₂═CH—CH₂— | (3-CF₃—Ph)—CH₂— |
| 453 | Cl—CH₂—CH₂—CH₂— | CH₂═CH—CH₂— | (3-CF₃—Ph)—CH₂— |
| 454 | CH₃— | CH≡C—CH₂— | (3-CF₃—Ph)—CH₂— |
| 455 | CH₃—CH₂— | CH≡C—CH₂— | (3-CF₃—Ph)—CH₂— |
| 456 | (CH₃)₂N— | CH≡C—CH₂— | (3-CF₃—Ph)—CH₂— |
| 457 | CH₃—CH₂—CH₂— | CH≡C—CH₂— | (3-CF₃—Ph)—CH₂— |
| 458 | (CH₃)₂CH— | CH≡C—CH₂— | (3-CF₃—Ph)—CH₂— |
| 459 | Cl—CH₂—CH₂—CH₂— | CH≡C—CH₂— | (3-CF₃—Ph)—CH₂— |
| 460 | CH₃— | CH₃—CH₂—CH(CH₃)— | (3-CF₃—Ph)—CH₂— |
| 461 | CH₃—CH₂— | CH₃—CH₂—CH(CH₃)— | (3-CF₃—Ph)—CH₂— |
| 462 | (CH₃)₂N— | CH₃—CH₂—CH(CH₃)— | (3-CF₃—Ph)—CH₂— |
| 463 | CH₃—CH₂—CH₂— | CH₃—CH₂—CH(CH₃)— | (3-CF₃—Ph)—CH₂— |
| 464 | (CH₃)₂CH— | CH₃—CH₂—CH(CH₃)— | (3-CF₃—Ph)—CH₂— |
| 465 | Cl—CH₂—CH₂—CH₂— | CH₃—CH₂—CH(CH₃)— | (3-CF₃—Ph)—CH₂— |

TABLE 19
Compounds represented by the Formula 1.19 where the combination of
the groups R₁ and R₃ corresponds to each row in table B.

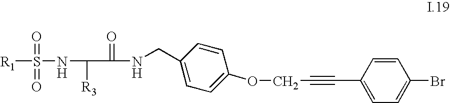

TABLE 20
Compounds represented by the Formula 1.20 where the combination of
the groups R₁ and R₃ corresponds to each row in table B.

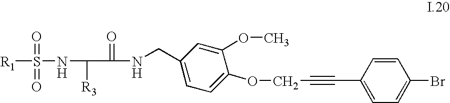

TABLE 21
Compounds represented by the Formula 1.21 where the combination of
the groups R₁ and R₃ corresponds to each row in table B.

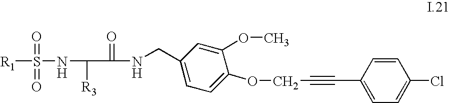

TABLE 22
Compounds represented by the Formula 1.22 where the combination of
the groups R₁ and R₃ corresponds to each row in table B.

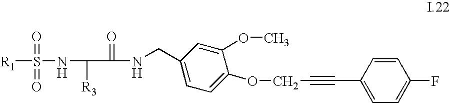

TABLE 23
Compounds represented by the Formula 1.23 where the combination of
the groups R₁ and R₃ corresponds to each row in table B.

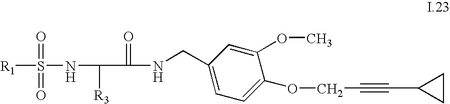

TABLE 24
Compounds represented by the Formula 1.24 where the combination of
the groups R₁ and R₃ corresponds to each row in table B.

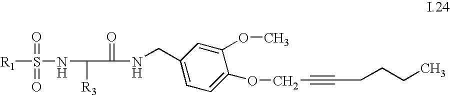

TABLE 25
Compounds represented by the Formula 1.25 where the combination of
the groups R₁ and R₃ corresponds to each row in table B.

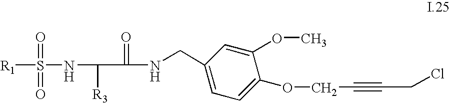

Table B

| No. | R₁ | R₃ |
|---|---|---|
| 001 | (CH₃—CH₂)₂N— | CH₃—CH₂— |
| 002 | CH₃—CH₂—(CH₃)N— | CH₃—CH₂— |
| 003 | 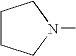 | CH₃—CH₂— |
| 004 | CH₃—(CH₂)₂—CH₂— | CH₃—CH₂— |
| 005 | (CH₃)₂CH—CH₂— | CH₃—CH₂— |
| 006 | CH₃—CH₂—(CH₃)CH— | CH₃—CH₂— |
| 007 | (CH₃)₃C— | CH₃—CH₂— |
| 008 | CH₂═CH— | CH₃—CH₂— |
| 009 | 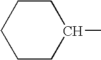 | CH₃—CH₂— |
| 010 | (CH₃—CH₂)₂N— | (CH₃)₂CH— |
| 011 | CH₃—CH₂—(CH₃)N— | (CH₃)₂CH— |
| 012 | 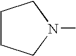 | (CH₃)₂CH— |
| 013 | CH₃—(CH₂)₂—CH₂— | (CH₃)₂CH— |
| 014 | (CH₃)₂CH—CH₂— | (CH₃)₂CH— |
| 015 | CH₃—CH₂—(CH₃)CH— | (CH₃)₂CH— |
| 016 | (CH₃)₃C— | (CH₃)₂CH— |
| 017 | CH₂═CH— | (CH₃)₂CH— |
| 018 | 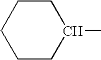 | (CH₃)₂CH— |
| 019 | (CH₃—CH₂)₂N— | CH₃—CH₂—CH₂ |
| 020 | CH₃—CH₂—(CH₃)N— | CH₃—CH₂—CH₂ |
| 021 | 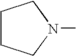 | CH₃—CH₂—CH₂ |
| 022 | CH₃—(CH₂)₂—CH₂— | CH₃—CH₂—CH₂ |
| 023 | (CH₃)₂CH—CH₂— | CH₃—CH₂—CH₂ |
| 024 | CH₃—CH₂—(CH₃)CH— | CH₃—CH₂—CH₂ |
| 025 | (CH₃)₃C— | CH₃—CH₂—CH₂ |
| 026 | CH₂═CH— | CH₃—CH₂—CH₂ |
| 027 | 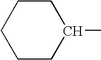 | CH₃—CH₂—CH₂ |
| 028 | (CH₃—CH₂)₂N— | CH₃—CH₂—(CH₃)CH— |
| 029 | CH₃—CH₂—(CH₃)N— | CH₃—CH₂—(CH₃)CH— |
| 030 | 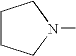 | CH₃—CH₂—(CH₃)CH— |
| 031 | CH₃—(CH₂)₂—CH₂— | CH₃—CH₂—(CH₃)CH— |
| 032 | (CH₃)₂CH—CH₂— | CH₃—CH₂—(CH₃)CH— |
| 033 | CH₃—CH₂—(CH₃)CH— | CH₃—CH₂—(CH₃)CH— |
| 034 | (CH₃)₃C— | CH₃—CH₂—(CH₃)CH— |
| 035 | CH₂═CH— | CH₃—CH₂—(CH₃)CH— |
| 036 | 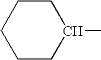 | CH₃—CH₂—(CH₃)CH— |
| 037 | (CH₃—CH₂)₂N— | 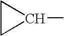 |
| 038 | CH₃—CH₂—(CH₃)N— | 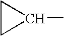 |
| 039 | 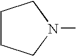 | 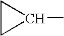 |
| 040 | CH₃—(CH₂)₂—CH₂— | 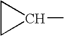 |
| 041 | (CH₃)₂CH—CH₂— | 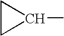 |
| 042 | CH₃—CH₂—(CH₃)CH— | 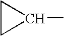 |
| 043 | (CH₃)₃C— | 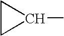 |
| 044 | CH₂═CH— | 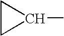 |
| 045 | 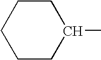 | 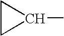 |
| 046 | CH₃— | HO—CH₂— |
| 047 | CH₃—CH₂— | HO—CH₂— |
| 048 | (CH₃)₂N— | HO—CH₂— |
| 049 | CH₃—CH₂—CH₂— | HO—CH₂— |
| 050 | (CH₃)₂CH— | HO—CH₂— |
| 051 | CH₃—CH₂—(CH₃)CH— | HO—CH₂— |
| 052 | CH₃— | HO—(CH₃)CH— |
| 053 | CH₃—CH₂— | HO—(CH₃)CH— |
| 054 | (CH₃)₂N— | HO—(CH₃)CH— |
| 055 | CH₃—CH₂—CH₂— | HO—(CH₃)CH— |
| 056 | (CH₃)₂CH— | HO—(CH₃)CH— |
| 057 | CH₃—CH₂—(CH₃)CH— | HO—(CH₃)CH— |
| 058 | CH₃— | (CH₃)₃C—O—(CH₃)H— |
| 059 | CH₃—CH₂— | (CH₃)₃C—O—(CH₃)CH— |
| 060 | (0H3)2N— | (0H3)30—O—(CH₃)OH— |
| 061 | CH₃—CH₂—CH₂— | (CH₃)₃C—O—(CH₃)CH— |
| 062 | (CH₃)₂CH— | (CH₃)₃C—O—(CH₃)CH— |
| 063 | CH₃—CH₂—(CH₃)CH— | (CH₃)₃C—O—(CH₃)CH— |
| 064 | CH₃— | CH₃—S—CH₂—CH₂— |
| 065 | CH₃—CH₂— | CH₃—S—CH₂—CH₂— |
| 066 | (CH₃)₂N— | CH₃—S—CH₂—CH₂— |
| 067 | CH₃—CH₂—CH₂— | CH₃—S—CH₂—CH₂— |
| 068 | (CH₃)₂CH— | CH₃—S——CH₂—CH₂— |
| 069 | CH₃—CH₂—(CH₃)CH— | CH₃—S—CH₂—CH₂— |
| 070 | CH₃— | HS—CH₂—CH₂— |
| 071 | CH₃—CH₂— | HS—CH₂—CH₂— |
| 072 | (CH₃)₂N— | HS—CH₂—CH₂— |
| 073 | CH₃—CH₂—CH₂— | HS—CH₂—CH₂— |
| 074 | (CH₃)₂CH— | HS—CH₂—CH₂— |
| 075 | CH₃—CH₂—(CH₃)CH— | HS—CH₂—CH₂— |
| 076 | CH₃— | (CH₃)₃C— |
| 077 | CH₃—CH₂— | (CH₃)₃C— |
| 078 | (CH₃)₂N— | (CH₃)₃C— |
| 079 | CH₃—CH₂—CH₂— | (CH₃)₃C— |
| 080 | (CH₃)₂CH— | (CH₃)₃C— |
| 081 | CH₃—CH₂—(CH₃)CH— | (CH₃)₃C— |

TABLE 26
Compounds represented by the Formula I.26 where the combination of
the group R₈ corresponds to each row in table C.

| | |
|---|---|
| | I.26 |
| 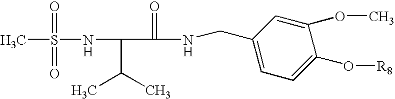 | |

TABLE 27
Compounds represented by the Formula I.27 where the combination of
the group R₈ corresponds to each row in table C.

| | |
|---|---|
| | I.27 |
| 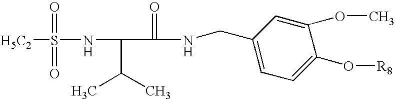 | |

TABLE 28
Compounds represented by the Formula I.28 where the combination of
the group R₈ corresponds to each row in table C.

| | |
|---|---|
| | I.28 |
| 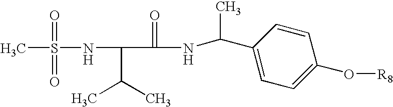 | |

TABLE 29
Compounds represented by the Formula I.29 where the combination of
the group R₈ corresponds to each row in table C.

| | |
|---|---|
| | I.29 |
| 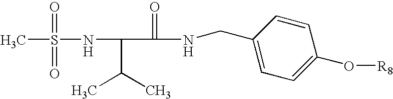 | |

TABLE 30
Compounds represented by the Formula I.30 where the combination of
the group R₈ corresponds to each row in table C.

| | |
|---|---|
| | I.30 |
| 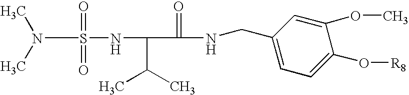 | |

TABLE C

| No. | R₈ |
|---|---|
| 001 | CH₃—CH₂—C(CH₃)H—C≡C—CH₂— |
| 002 | (CH₃)₃C—C≡C—CH₂— |
| 003 | (CH₃)₂CH—CH₂—C≡C—CH₂— |
| 004 | CH₃—(CH₂)₄—C≡C—CH₂— |
| 005 | 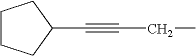 |
| 006 | 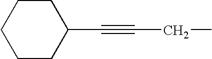 |
| 007 | (4-CH₃—Ph)—C≡C—CH₂— |
| 008 | (2-Cl—Ph)—C≡C—CH₂— |
| 009 | (3-Cl—Ph)—C≡C—CH₂— |
| 010 | (3-Br—Ph)—C≡C—CH₂— |
| 011 | (3-F—Ph)—C≡C—CH₂— |
| 012 | (3-CH₃—Ph)—C≡C—CH₂— |
| 013 | (2,4-di-Cl—Ph)—C≡C—CH₂— |
| 014 | (3,4-di-Cl—Ph)—C≡C—CH₂— |
| 015 | (3,4-di-F—Ph)—C≡C—CH₂— |
| 016 | (3-CH₃-4-Cl—Ph)—C≡C—CH₂— |
| 017 | (3-CH₃-4-Br—Ph)—C≡C—CH₂— |
| 018 | (3-CF₃—Ph)—C≡C—CH₂— |
| 019 | (4-CF₃O—Ph)—C≡C—CH₂— |
| 020 | (4-Et—Ph)—C≡C—CH₂— |
| 021 | [4-(CH₃)₃C—Ph]—C≡C—CH₂— |
| 022 | (4-CH₂═CH—Ph)—C≡C—CH₂— |
| 023 | (4-CH≡C—Ph)—C≡C—CH₂— |
| 024 | (4-CH₃—CO—Ph)—C≡C—CH₂— |
| 025 | (4-CH₃OOC—Ph)—C≡C—CH₂— |
| 026 | (4-CH₃O—Ph)—C≡C—CH₂— |
| 027 | 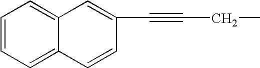 |
| 028 | 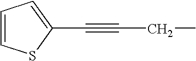 |
| 029 | 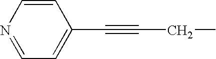 |
| 030 | 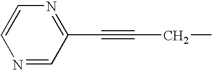 |
| 031 | Ph—(CH₃)CH— |
| 032 | Ph—(CH₃)₂C— |
| 033 | (3-F—Ph)—CH₂— |
| 034 | (4-Br—Ph)—CH₂— |
| 035 | (4-J-Ph)—CH₂— |
| 036 | (4-CH₃—Ph)—CH₂— |
| 037 | (4-CH₃O—Ph)—CH₂— |
| 038 | (4-CF₃O—Ph)—CH₂— |
| 039 | (4-F—Ph)—CH₂— |
| 040 | (2,4-di-F—Ph)—CH₂— |
| 041 | (3,4-di-F—Ph)—CH₂— |
| 042 | (3,4-di-Br—Ph)—CH₂— |
| 043 | (3,4-di-Cl—Ph)—CH₂— |
| 044 | (3-Cl-4-CH₃—Ph)—CH₂— |
| 045 | (3-CH₃-4-Cl—Ph)—CH₂— |
| 046 | (2,4,5-tri-Cl—Ph)—CH₂— |
| 047 | 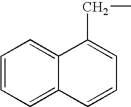 |
| 048 | 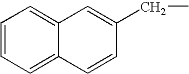 |
| 049 | 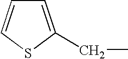 |
| 050 | 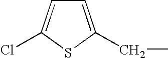 |
| 051 | 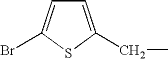 |
| 052 | 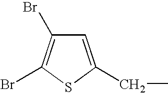 |
| 053 | 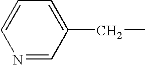 |
| 054 | 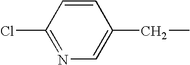 |
| 055 | 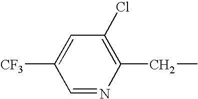 |
| 056 | 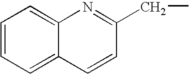 |
| 057 | Ph—CH═CH—CH₂— |
| 058 | (4-Cl—Ph)—CH═CH—CH₂— |
| 059 | (4-F—Ph)—CH═CH—CH₂— |
| 060 | (4-Br—Ph)—CH═CH—CH₂— |
| 061 | (3-CF₃—Ph)—CH═CH-₂- |
| 062 | Ph—CH₂—CH₂— |
| 063 | Ph—(CH₂)₂—CH₂— |
| 064 | (4-Cl—Ph)—(CH₂)₂—CH₂— |
| 065 | (4-Br—Ph)—(CH₂)₂—CH₂— |
| 066 | Ph—O—CH₂—CH₂— |
| 067 | (4-Cl—Ph)—O—CH₂—CH₂— |
| 068 | (4-F—Ph)—O—CH₂—CH₂— |
| 069 | (4-Br—Ph)—O—CH₂—CH₂— |
| 070 | (3-F—Ph)—O—CH₂—CH₂— |
| 071 | (3,4-di-Cl—Ph)—O—CH₂—CH₂— |
| 072 | (4-Cl—Ph)—O—(CH₃)CH—CH₂— |
| 073 | (3,4-di-Cl—Ph)—O—(CH₃)CH—CH₂— |
| 074 | Ph—N(CH₃)—CH₂—CH₂— |
| 075 | Ph—N(CH₂—CH₃)—CH₂—CH₂— |
| 076 | Ph—S—CH₂—CH₂— |
| 077 | (4-Cl—Ph)—S—CH₂—CH₂— |

Formulations may be prepared analogously to those described in, for example, WO 95/30651.

### BIOLOGICAL EXAMPLES

D-1: Action Against Plasmopara viticola (downy mildew) on Vines

5 week old grape seedlings cv. Gutedel are treated with the formulated test compound in a spray chamber. One day after application grape plants are inoculated by spraying a sporangia suspension (4×10⁴ sporangia/ml) on the lower leaf side of the test plants. After an incubation period of 6 days at +21° C. and 95% r. h. in a greenhouse the disease incidence is assessed.

Compounds of Tables 1 to 30 exhibit a good fungicidal action against Plasmopara viticola on vines. Compounds A1.1, A1.2, A1.3, A1.4, A1.5 and A1.6 at 200 ppm inhibit fungal infestation in this test to a least 80%, while under the same conditions untreated control plants are infected by the phytopathogenic fungi to over 80%.

D-2: Action Against Phytophthora (late blight) on Tomato Plants

3 week old tomato plants cv. Roter Gnom are treated with the formulated test compound in a spray chamber. Two day after application the plants are inoculated by spraying a sporangia suspension (2×10⁴ sporangia/ml) on the test plants. After an incubation period of 4 days at +18° C. and 95% r. h. in a growth chamber the disease incidence is assessed. Compounds of Tables 1 to 30 exhibit a long-lasting effect against fungus infestation. Compounds A1.1, A1.2, A1.3, A1.4, A1.5 and A1.6 at 200 ppm inhibit fungal infestation in this test to a least 80%, while under the same conditions untreated control plants are infected by the phytopathogenic fungi to over 80%.

D-3: Action Against Phytophthora (late blight) on Potato Plants

5 week old potato plants cv. Bintje are treated with the formulated test compound in a spray chamber. Two day after application the plants are inoculated by spraying a sporangia suspension (14×10⁴ sporangia/ml) on the test plants. After an incubation period of 4 days at +18° C. and 95% r. h. in a growth chamber the disease incidence is assessed. Fungal infestation is effectively controlled with compounds of Tables 1 to 30. Compounds A1.1, A1.2, A1.3, A1.4, A1.5 and A1.6 at 200 ppm inhibit fungal infestation in this test to a least 80%, while under the same conditions untreated control plants are infected by the phytopathogenic fungi to over 80%.

What is claimed is:

1. α-Sulfin- and α-sulfonamino acid amides of formula I 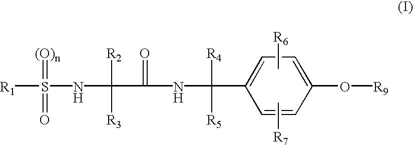
including the optical isomers thereof and mixtures of such isomers, wherein
n is a number zero or one;
R₁ is C₁-C₁₂alkyl, C₁-C₁₂alkyl substituted with C₁-C₄alkoxy, C₁-C₄alkylthio, C₁-C₄alkylsulfonyl, C₃-C₈cycloalkyl, cyano, C₁-C₆alkoxycarbonyl, C₃-C₆alkenyloxycarbonyl or C₃-C₆alkynyloxycarbonyl; C₃-C₈cycloalkyl; C₂-C₁₂alkenyl; C₂-C₁₂alkynyl; C₁-C₁₂halogenalkyl; or a group NR₉R₁₀ wherein R₉ and R₁₀ are independently of the other C₁-C₆alkyl, or together are tetra- or penta-methylene;
R₂ and R₃ are each independently hydrogen; C₁-C₈alkyl; C₁-C₈alkyl substituted with hydroxy, mercapto, C₁-C₄alkoxy or C₁-C₄alkylthio; C₃-C₈alkenyl; C₃-C₈alkynyl; C₃-C₈cycloalkyl; C₃-C₈cycloalkyl-C₁-C₄alkyl; or the two groups R₂ and R₃ together with the carbon atom to which they are bonded form a three to eight-membered hydrocarbon ring;
R₄ and R₅ are each independently hydrogen or C₁-C₄alkyl;
R₆ and R₇ are independently of each other hydrogen or an organic radical,
R₈ is either hydrogen 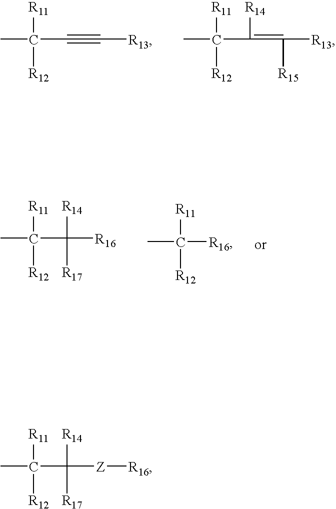
wherein
R₁₁, R₁₂, R₁₃, R₁₅ and R₁₇ are each independently hydrogen or C₁-C₄alkyl,
R₁₃ is C₄-C₁₂alkyl; C₁-C₁₂halogenalkyl; C₃-C₈cycloalkyl; optionally substituted aryl or optionally substituted heteroaryl,
R₁₆ is optionally substituted aryl or optionally substituted heteroaryl; and
Z is oxygen, sulfur —CR₁₈R₁₉— or —NR₂₀—, wherein R₁₈, R₁₉ and R₂₀ independently of each other are hydrogen or C₁-C₄alkyl.

2. A compound according to claim 1 wherein n is one.

3. A compound of formula I according to claim 1 wherein
R₁ is C₁-C₁₂alkyl, C₁-C₁₂alkyl substituted with C₁-C₄alkoxy, C₁-C₄alkylthio, or C₁-C₄alkylsulfonyl; C₃-C₈cycloalkyl; C₂-C₁₂alkenyl; C₂-C₁₂alkynyl; C₁-C₁₂halogenalkyl; or a group NR₉R₁₀ wherein R₉ and R₁₀ are each independently of the other hydrogen or C₁-C₆alkyl, or together are tetra- or penta-methylene.

4. A compound of formula I according to claim 1 wherein
R₂ is hydrogen and R₃ is C₁-C₈alkyl, C₁-C₈alkyl optionally substituted by hydroxy, C₁-C₄-alkoxy, mercapto or C₁-C₄alkylthio; C₃-C₈alkenyl; C₃-C₈alkynyl; C₃-C₈cycloalkyl or C₃-C₈cycloalkyl-C₁-C₄alkyl.

5. A compound of formula I according to claim 1 wherein R₄ is hydrogen, methyl or ethyl.

6. A compound of formula I according to claim 1 wherein R₅ is hydrogen or methyl.

7. A compound of formula I according to claim 1 wherein
R₆ is hydrogen, C₁-C₈alkyl, C₂-C₈alkenyl, C₂-C₈alkynyl, C₃-C₈cycloalkyl, C₃-C₈cycloalkyl-C₁-C₄alkyl, C₁-C₈alkylthio, C₁-C₈alkylsulfonyl, C₁-C₈alkoxy, C₃-Calkenyloxy, C₃-C₈alkynyloxy, C₃-C₈cycloalkoxy, C₁-C₈alkoxy-C₁-C₄alkyl, C₁-C₈alkoxycarbonyl, C₃-C₈alkenyloxycarbonyl, C₃-C₈alkynyloxycarbonyl, C₁-C₈alkanoyl, C₁-C₈dialkylamino, C₁-C₈alkylamino wherein in each of the above radicals the alkyl, alkenyl, alkynyl or cycloalkyl groups may be partially or fully halogenated; carboxy, formyl, halogen, nitro, cyano, hydroxy or amino; or

8. A compound of formula I according to claim 1 wherein
R₇ is hydrogen, C₁-C₈alkyl, C₂-C₈alkenyl, C₂-C₈alkynyl, C₃-C₈cycloalkyl, C₃-C₈cycloalkyl-C₁-C₄-alkyl, C₁-C₈alkylthio, C₁-C₈alkylsulfonyl, C₁-C₈alkoxy, C₃-C₈alkenyloxy, C₃-C₈alkynyloxy, C₃-C₈cycloalkoxy, C₁-C₈alkoxy-C₁-C₄alkyl, C₁-C₈alkoxycarbonyl, C₃-C₈alkenyloxycarbonyl, C₃-C₈alkynyloxycarbonyl, C₁-C₈alkanoyl, C₁-C₈dialkylamino, C₁-C₈alkylamino wherein in each of the above radicals the alkyl, alkenyl, alkynyl or cycloalkyl groups may be partially or fully halogenated; carboxy, formyl, halogen, nitro, cyano, hydroxy or amino; or

9. A compound of formula I according to claim 1 wherein
R₁₁, R₁₂, R₁₄, R₁₅ and R₁₇ are each independently hydrogen or methyl.

10. A compound of formula I according to claim 1 wherein
R₁₃ is C₄-C₁₂alkyl; C₁-C₁₂halogenalkyl; C₃-C₈cycloalkyl; optionally substituted aryl or optionally substituted heteroaryl consisting of one or two condensed five or six membered rings with 1 to 4 identical or different heteroatoms selected from oxygen, nitrogen or sulfur.

11. A compound of formula I according to claim 1 wherein
R₁₆ is optionally substituted aryl or optionally substituted heteroaryl consisting of one or two condensed five or six membered rings with 1 to 4 identical or different heteroatoms selected from oxygen, nitrogen or sulfur.

12. A compound of formula I according to claim 1 wherein Z is oxygen, sulfur or —CH ₂—.

13. A compound of formula I according to claim 1 wherein
n is one;
R₁ is C₁-C₄alkyl, vinyl; C₁-C₄halogenalkyl; or dimethylamino;
R₂ is hydrogen and R₃ is isopropyl;
R₄, R₅, R₇, R₁₁, R₁₂, R₁₄, R₁₅ and R₁₇ are each hydrogen;
R₆ is hydrogen, C₁-C₄alkyl, C₁-C₄alkoxy, halogen or cyano;
R₁₃ is C₄-C₈alkyl; C₁-C₆halogenalkyl; C₃-C₈cycloalkyl; phenyl, pyridyl, with each of the aromatic ring being optionally substituted by 1 to 3 substituents selected from the group consisting of C₁-C₈alkyl, C₁-C₈halogenalkyl, C₁-C₈alkoxy, C₁-C₈halogenalkoxy, C₁-C₈alkylthio, C₁-C₈halogenalkylthio, halogen, cyano, nitro and C₁-C₈alkoxycarbonyl;
R₁₆ is phenyl, pyridyl, with each of the aromatic ring being optionally substituted by 1 to 3 substituents selected from the group consisting of C₁-C₈alkyl, C₁-C₈halogenalkyl, C-C₈alkoxy, C₁-C₈halogenalkoxy, C₁-C₈alkylthio, C₁-C₈halogenalkylthio, halogen, cyano, nitro and C₁-C₈alkoxycarbonyl; and
Z is oxygen.

14. A process for the preparation of a compound of formula I according to claim 1, which comprises reacting
a) an amino acid of formula II or a carboxy-activated derivative thereof 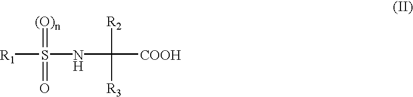
wherein R₁, n, R₂ and R₃ are as defined for formula I is reacted with an amine of formula III 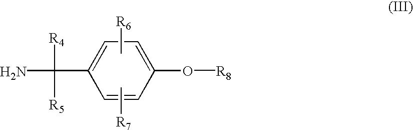
wherein R₄, R₅, R₆, R₇ and R₈ are as defined for formula I optionally in the presence of a base and optionally in the presence of a diluting agent, or
b) an amino acid derivative of formula VI 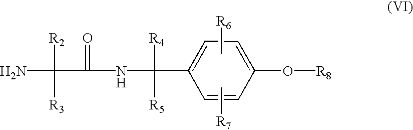
wherein R₂, R₃, R₄, R₅, R₆, R₇ and R₈ are as defined for formula I with a sulfonyl halide or a sulfinyl halide of formula V 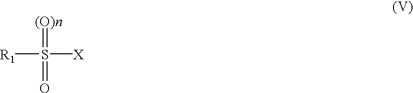
wherein R₁ and n are as defined for formula I and X is halide, preferentially chlorine or bromine, or
c) a phenol of formula VII 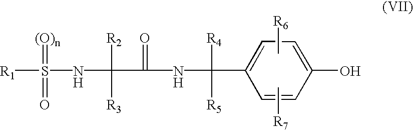
wherein R₁, n, R₂, R₃, R₄, R₅, R₆ and R₇ are as defined for formula I with a compound of formula VIII
Y—R₈ (VIII)
wherein R₈ is as defined for formula I and Y is a leaving group like a halide such as a chloride or bromide or a sulfonic ester such as a tosylate, mesylate or triflate.

15. A process for the preparation of a compound of formula Ia 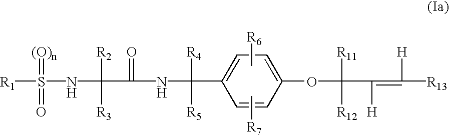
wherein R₁, n, R₂, R₃, R₄, R₅ , R₆, R₇, R₁₁, R₁₂ and R₁₃ are defined in claim 1 for formula which comprises reacting a compound of formula IX 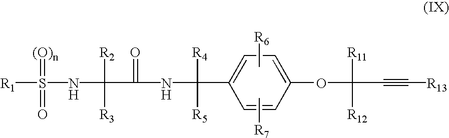
wherein R₁, n, R₂, R₃, R₄, R₅, R₆, R₇, R₁₁, R₁₂ and R₁₃ are defined for formula I with hydrogen.

16. A composition for controlling and protecting against phytopathog enic microorganisms, comprising a compound of formula I according to claim 1 as active ingredient together with a suitable carrier.

17. The use of a compound of formula I according to claim 1 in protecting plants against infestation by phytopathogenic microorganisms.

18. A method of controlling and preventing an infestation of crop plants by phytopathogenic microorganisms, preferably fungal organisms, which comprises the application of a compound of formula I according to claim 1 as active ingredient to the plant, to parts of plants or to the locus thereof.
